(19) United States
(12) Patent Application Publication
(10) Pub. No.: US 2025/0387222 A1
(43) Pub. Date: Dec. 25, 2025

(54) DELIVERY DEVICE

(71) Applicant: Alcon Inc., Fribourg (CH)

(72) Inventors: Robertus Christophorus Maria Bosco MEIJER, Falkirk (GB); James Christopher Charles BEMBRIDGE, Cambridgeshire (GB); James Kenneth DOUIE, Cambrigeshire (GB); Samuel James HAIGH, Cambridge (GB); Abul Hasnath Mohammed Shafiul ALAM, Hertfordshire (GB)

(21) Appl. No.: 19/231,819

(22) Filed: Jun. 9, 2025

Related U.S. Application Data
(60) Provisional application No. 63/662,029, filed on Jun. 20, 2024.

(51) Int. Cl. A61F 2/16

(52) U.S. Cl. CPC A61F 2/167

(57) ABSTRACT

A delivery device includes a delivery portion with a nozzle for delivery of an object and/or material through the nozzle, a barrel coupled to the delivery portion, and a plunger received within the barrel. The plunger may include a plunger tip to engage with the object and/or material, for delivery thereof through the nozzle; a plunger driver to drive the plunger tip to deliver the object and/or material; a stored energy source providing a drive force to the plunger driver when released for driving the plunger driver and corresponding movement of the plunger tip; a release mechanism for providing a controlled release of the stored energy; and an actuator to control the release mechanism to induce the controlled release of the stored energy. The plunger tip driver may operate between: the plunger driver is disengaged from the plunger tip; and the plunger driver is engaged with the plunger tip element.

### TECHNICAL FIELD

The present disclosure relates to a delivery device for deploying a substance or object, and to associated methods. The present disclosure has particular, but not exclusive, relevance to a plunger apparatus for a delivery device for implantation of an intraocular lens (IOL) into an eye of a subject and corresponding methods for manufacturing and using such apparatus.

### BACKGROUND

There are many medical and non-medical procedures in which a medical practitioner or other operator is expected to dispense a viscous substance, or to deploy a small object (often held in a viscous medium), in a precise and controlled manner. Such procedures typically utilize a highly skilled operator to apply a relatively high force, often on a routine basis, while maintaining a high level of accuracy.

One such application is the implantation of an intraocular lens (IOL) into the eye of a patient as part of a procedure for treating medical conditions such as cataracts or myopia.

To support such procedures, IOL injectors or insertion devices have developed that allow a surgeon to insert an IOL, into the eye of a patient, through an incision that is considerably narrower than the width of the lens. These devices typically fold the lens into a smaller size by advancing it, with a plunger, through a narrow nozzle. For clinical reasons a viscous agent, known as a viscoelastic or ‘ophthalmic viscosurgical device’ (‘OVD’), is added as the lens is offered up to the nozzle.

The friction between the lens and the nozzle of an IOL injector can be high and, depending on the size and type of the lens, the delivery plunger may therefore have to be driven with a correspondingly high force by the responsible practitioner. The delivery is typically executed in a very controlled manner to avoid a quick release of stored energy at the moment the lens exits the nozzle and enters the eye.

Existing devices typically come in the form of syringe-like, manually driven, delivery devices. However these can be difficult for an operator to control, particularly when the IOL exits the injector, because of the sharp drop in the force utilized for delivery, from the relatively high level used to push the lens through the nozzle, to almost zero immediately after the lens has left the nozzle. As the force drops suddenly, there is a risk that the operator may lose control of the device and, as a result, that the nozzle, which rests in a minute incision in the eye, causes an ocular injury.

While the risk associated with the step-change in application force may be ameliorated in manually operated screw based IOL delivery devices, these devices tend to deliver lenses relatively slowly. Moreover, the rotational movements made by the operator to operate a screw based or similar device can cause undesirable movement of the nozzle with an associated risk that eye tissue can be torn adjacent the incision.

In general, manually operated screw-based devices also utilize two hands for operation. However, there is a preference amongst some surgeons to have one hand free to help steady the patient's eye during lens implantation and to guide the emerging lens into location. Electrically driven, hydraulically driven, compressed gas driven, and spring driven types of delivery mechanisms have been contemplated and introduced into the marketplace. While these have the benefit that they can free a surgeon's hand to assist in the insertion procedure, existing devices tend to be heavy, poorly balanced, mechanically complex, and expensive.

It will be appreciated that analogous issues can arise with any similar delivery device for other applications in which an operator is expected to dispense a viscous substance, or to deploy a device held in a viscous medium, in a precise and controlled manner. Even where the force or precision utilized is not particularly great, there is a risk of repetitive strain injury in any similar procedure where an operator uses such a delivery device routinely.

As with any such delivery device, especially for medical applications, it may be desirable for the device to meet one or more safety and/or ease of use requests. For example, the device may be reliable, intuitive, and simple to use, with little or no requirements for operator training or skill-based judgements to be made during operation.

Moreover the device may allow a viscous substance to be dispensed (or a small object held in a viscous medium to be deployed) using a procedure involving one or more smooth, human controllable, actions—avoiding or at least minimizing, for example, friction induced vibration or ‘judder’. In some embodiments, the device may be safe and reliable to use with little or no risk of inadvertent premature activation that could lead to injury (e.g., to a patient's eye) or procedure failure. In some embodiments, the device may be capable of being subject to prolonged storage without significant deterioration of various components over time, e.g., due to any inherent internal stresses that the components may be subject to. Similarly, the device may be robust and capable of withstanding the stresses inherent to typical transportation (for example, with little or no risk of movement of internal components in a manner which could jeopardize successful operation of the device, e.g., as a result of jamming).

The disclosure may, in some embodiments, provide an improved delivery device and an associated method, for example a delivery device with an improved plunger drive mechanism that overcomes or at least partially ameliorates one or more of the above issues and/or that has features aimed at meeting or at least partially addressing one or more of the above issues, and one or more associated methods for manufacturing or using such a delivery device.

### SUMMARY

Various aspects of the present disclosure are set out in the appended independent claims. Other features are also recited in the dependent claims and throughout the specification.

In one aspect there is provided a delivery device for deploying at least one object and/or material, the delivery device comprising: a delivery portion comprising a nozzle, the delivery portion being arranged for delivery of the at least one object and/or material through the nozzle; a barrel portion coupled to the delivery portion; and a plunger portion at least partially received within the barrel portion, the plunger portion comprising: a plunger tip element configured for movement in an axial direction relative to the barrel portion to engage with the at least one object and/or material, for delivery of the at least one object and/or material from the nozzle; a plunger driver configured for movement in the axial direction relative to the barrel portion, and for engagement with the plunger tip element, to drive corresponding movement of the plunger tip element to deliver the at least one object and/or material; a source of stored energy coupled to the plunger driver, the source of stored energy being arranged for providing a drive force to the plunger driver, when the stored energy is released, for driving the movement of the plunger driver and corresponding movement of the plunger tip element; a release mechanism for providing a controlled release of the stored energy for providing the drive force for driving the movement of the plunger driver; and an actuator configured for operation by an operator of the delivery device to control the release mechanism to induce the controlled release of the stored energy; wherein herein the plunger tip element and the plunger driver are mutually configured for operation between: a disengaged arrangement in which the plunger driver is disengaged from the plunger tip element for the purpose of driving the corresponding movement of the plunger tip element; and an engaged arrangement in which the plunger driver is engaged with the plunger tip element for the purpose of driving the corresponding movement of the plunger tip element.

In one aspect there is provided a method of using the delivery device, the method comprising: operating the delivery device to change an arrangement of the plunger tip element and plunger driver from the disengaged arrangement to the engaged arrangement; and operating the actuator to control the release mechanism to induce the controlled release of the stored energy to deliver the at least one object and/or material through the nozzle.

In one aspect there is provided a method of manufacturing a delivery device, the method comprising: providing the delivery portion, the barrel portion, and the plunger portion; and assembling the delivery portion, the barrel portion, and the plunger portion to form the delivery device.

The actuator may be configured for operation to change an arrangement of the plunger tip element and plunger driver from the disengaged arrangement to the engaged arrangement. The actuator may be configured for operation to move the plunger tip element from a first position relative to the barrel portion to a second position relative to the barrel portion, whereby to change the arrangement of the plunger tip element and plunger driver from the disengaged arrangement to the engaged arrangement. The actuator may be configured for operation to change the arrangement of the plunger tip element and plunger driver from the disengaged arrangement to the engaged arrangement by movement of the actuator in the axial direction, relative to the barrel portion, toward the nozzle.

The release mechanism may be configured for operation between: a first configuration in which the release mechanism is inhibited from providing the controlled release of the stored energy to provide the drive force for driving the movement of the plunger driver; and a second configuration in which the release mechanism is released from inhibition from providing the controlled release of the stored energy, whereby to allow for the controlled release of the stored energy to provide the drive force for driving the movement of the plunger driver.

The delivery device may further comprise a locking mechanism having a locking element configured for movement, when the plunger tip element and the plunger driver are in the disengaged arrangement, to change a configuration of the delivery device between: a locking configuration in which the locking element engages with both the plunger tip element and the plunger driver to inhibit movement of the plunger tip element and the plunger driver in the axial direction; and an unlocked configuration in which the locking element is disengaged from both the plunger tip element and the plunger driver, to release the plunger tip element and the plunger driver from inhibition by the locking element, whereby to allow for movement of the plunger tip element and the plunger driver in the axial direction.

The actuator may be configured for operation by an operator through a sequence of operations to control operation of the delivery device to perform delivery of the at least one object and/or material through the nozzle, the sequence of operations including a plurality of different types of actuator movement, wherein the delivery device further comprises an indicator mechanism configured to: display a first indication of a first type of actuator movement of the plurality of different types of actuator movement, utilized in a first operation of the sequence of operations, and to display a second indication of a second different type of actuator movement of the plurality of different types of actuator movement, utilized in a second operation of the sequence of operations, following the first operation, as a result of movement of the actuator to perform the first operation of the sequence of operations.

In one aspect there is provided a delivery device for deploying at least one object and/or material, the delivery device comprising: a delivery portion comprising a nozzle, the delivery portion being arranged for delivery of the at least one object and/or material through the nozzle; a barrel portion coupled to the delivery portion; and a plunger portion at least partially received within the barrel portion, the plunger portion comprising: a plunger tip element configured for movement in an axial direction relative to the barrel portion to engage with the at least one object and/or material, for delivery of the at least one object and/or material from the nozzle; a plunger driver configured for movement in the axial direction relative to the barrel portion, and for engagement with the plunger tip element, to drive corresponding movement of the plunger tip element to deliver the at least one object and/or material; a source of stored energy coupled to the plunger driver, the source of stored energy being arranged for providing a drive force to the plunger driver, when the stored energy is released, for driving the movement of the plunger driver and corresponding movement of the plunger tip element; a release mechanism for providing a controlled release of the stored energy for providing the drive force for driving the movement of the plunger driver; and an actuator configured for operation by an operator of the delivery device to control the release mechanism to induce the controlled release of the stored energy; wherein the release mechanism and the plunger driver are mutually configured for operation between: a first configuration in which the release mechanism is inhibited from providing the controlled release of the stored energy to provide the drive force for driving the movement of the plunger driver; and a second configuration in which the release mechanism is released from inhibition from providing the controlled release of the stored energy, whereby to allow for the controlled release of the stored energy to provide the drive force for driving the movement of the plunger driver.

In one aspect there is provided a method of using the delivery device, the method comprising: operating the delivery device to change a configuration of the release mechanism and the plunger driver from the first configuration to the second configuration; and operating the actuator to control the release mechanism to induce the controlled release of the stored energy to deliver the at least one object and/or material through the nozzle.

In one aspect there is provided a method of manufacturing a delivery device, the method comprising: providing the delivery portion, the barrel portion, and the plunger portion; and assembling the delivery portion, the barrel portion, and the plunger portion to form the delivery device.

The actuator may be configured for operation to change a configuration of the release mechanism and plunger driver from the first configuration to the second configuration. The actuator may be configured for operation to change the configuration of the release mechanism and plunger driver from the first configuration to the second configuration by rotation of the actuator about a longitudinal axis of the barrel portion. The actuator may be configured for operation by an operator of the delivery device to control the release mechanism to induce the controlled release of the stored energy, when the release mechanism and the plunger driver are in the second configuration, by movement of the actuator in the axial direction, relative to the barrel portion, toward the nozzle.

The release mechanism may comprise a drive portion and a rotatable element that are mutually configured for rotation of the rotatable element relative to the drive portion, about an axis of rotation that is parallel to the axial direction, whereby rotation of the rotatable element through a given cumulative rotational displacement provides the controlled release of the stored energy for driving the movement of the plunger driver, in the axial direction relative to the barrel portion, under the force provided by the source of stored energy, for a longitudinal displacement that is dependent on the cumulative rotational displacement. The actuator may be configured for axial movement, by the operator, relative to the barrel portion to induce corresponding axial movement of the drive portion, wherein the corresponding axial movement of the drive portion induces the rotation of the rotatable element relative to the drive portion to provide the controlled release of the stored energy.

The delivery device may further comprise at least one element for providing a preconfigured resistance between the drive portion and the barrel portion for resisting axial movement of the drive portion relative to the barrel portion whereby the drive portion is moveable by an operator applying a substantially uniform force that is sufficient for overcoming the preconfigured resistance. The resistance element may comprise a spring.

The drive portion and the rotatable element may each have a respective threaded surface, wherein the threaded surfaces of the drive portion and the rotatable element are configured for mutual engagement whereby the rotatable element is configured for the rotation relative to the drive portion, as the drive portion is moved in the axial direction by the actuator, whereby to provide the controlled release of the stored energy. The threaded surfaces of the drive portion and the rotatable element may be mutually configured such that the rotatable element is back-drivable with respect to the drive portion. The drive portion may comprise a tubular sleeve, and the tubular sleeve and rotatable element may be mutually configured for rotation of the rotatable element within the tubular sleeve. The plunger driver and the rotatable element may each have a respective threaded surface, wherein the threaded surfaces of the plunger driver and the rotatable element are configured for mutual engagement whereby the rotatable element is configured for rotation relative to the plunger driver as the rotatable element rotates relative to the sleeve to provide the controlled release of the stored energy. The threaded surfaces of the plunger driver and the rotatable element may be mutually configured such that the rotatable element is back-drivable with respect to the plunger driver. The drive portion may comprise a cylindrical threaded portion, and the cylindrical threaded portion and rotatable element may be mutually configured for rotation of the rotatable element about the cylindrical portion.

The source of stored energy may be a source of stored mechanical energy. The source of stored mechanical energy may comprise a spring that is arranged for storing elastic potential energy prior to operation of the delivery device for providing the drive force for driving the movement of the plunger driver. The spring may be arranged to be in a compressed state for storing the elastic potential energy prior to operation of the delivery device for providing the drive force for driving movement of the plunger driver.

The drive portion and the rotatable element may be formed of materials that provide a kinetic friction coefficient of between 0.4 and 0.8 inclusive at an interface between the drive portion and the rotatable element. The drive portion and the rotatable element may be formed of materials that provide a static friction coefficient at an interface between the plunger driver and the rotatable element that is equal to, or less than but within 10% of, the kinetic friction coefficient at that interface.

The plunger driver and the rotatable element may be formed of materials that provide a kinetic friction coefficient of between 0.2 and 0.5 inclusive at an interface between the plunger driver and the rotatable element. The plunger driver and the rotatable element may be formed of materials that provide a static friction coefficient at an interface between the plunger driver and the rotatable element that is less than but within 10% of the kinetic friction coefficient at that interface.

The drive portion may be configured for slidable movement within the barrel portion, and the drive portion and barrel portion may be formed of materials that provide a kinetic friction coefficient of between 0.4 and 0.8 inclusive at an interface between the drive portion and barrel portion. The drive portion and barrel portion may be formed of materials that provide a static friction coefficient at an interface between the drive portion and barrel portion that is equal to, or less than but within 10% of, the kinetic friction coefficient at that interface.

In one aspect there is provided a delivery device for deploying at least one object and/or material, the delivery device comprising: a delivery portion comprising a nozzle, the delivery portion being arranged for delivery of the at least one object and/or material through the nozzle; a barrel portion coupled to the delivery portion; and a plunger portion at least partially received within the barrel portion, the plunger portion comprising: a plunger tip element configured for movement in an axial direction relative to the barrel portion to engage with the at least one object and/or material, for delivery of the at least one object and/or material from the nozzle; a plunger driver configured for movement in the axial direction relative to the barrel portion, and for engagement with the plunger tip element, to drive corresponding movement of the plunger tip element to deliver the at least one object and/or material; a source of stored energy coupled to the plunger driver, the source of stored energy being arranged for providing a drive force to the plunger driver, when the stored energy is released, for driving the movement of the plunger driver and corresponding movement of the plunger tip element; a release mechanism for providing a controlled release of the stored energy for providing the drive force for driving the movement of the plunger driver; and an actuator configured for operation by an operator of the delivery device to control the release mechanism to induce the controlled release of the stored energy; and wherein the delivery device further comprises a locking mechanism having a locking element configured for movement to change a configuration of the delivery device between: a locking configuration in which the locking element engages with both the plunger tip element and the plunger driver to inhibit movement of the plunger tip element and the plunger driver in the axial direction; and an unlocked configuration in which the locking element is disengaged from both the plunger tip element and the plunger driver to release the plunger tip element and the plunger driver from inhibition by the locking element, whereby to allow for movement of the plunger tip element and the plunger driver in the axial direction.

In one aspect there is provided a method of using the delivery device, the method comprising: operating the locking mechanism to change a configuration of the delivery device from the locking configuration to the unlocked configuration; and operating the actuator to control the release mechanism to induce the controlled release of the stored energy to deliver the at least one object and/or material through the nozzle.

In one aspect there is provided a method of manufacturing a delivery device, the method comprising: providing the delivery portion, the barrel portion, the plunger portion, and the locking mechanism; and assembling the delivery portion, the barrel portion, the plunger portion, and the locking mechanism to form the delivery device.

The locking mechanism may comprise a button coupled to the locking element, and the locking mechanism may be configured for movement of the locking element, by the operator pushing the button, to change the configuration of the delivery device from the locking configuration to the unlocked configuration. The locking element may be configured for removal from the barrel portion, by the operator, to change the configuration of the delivery device from the locking configuration to the unlocked configuration.

In one aspect there is provided a delivery device for deploying at least one object and/or material, the delivery device comprising: a delivery portion comprising a nozzle, the delivery portion being arranged for delivery of the at least one object and/or material through the nozzle; a barrel portion coupled to the delivery portion; and a plunger portion at least partially received within the barrel portion, the plunger portion comprising a plunger tip element configured for movement in an axial direction relative to the barrel portion to engage with the at least one object and/or material, for delivery of the at least one object and/or material from the nozzle, and an actuator configured for operation by an operator through a sequence of operations to control movement of the plunger tip element to perform delivery of the at least one object and/or material through the nozzle, the sequence of operations including a plurality of different types of actuator movement; wherein the delivery device further comprises an indicator mechanism configured to: display a first indication of a first type of actuator movement of the plurality of different types of actuator movement, utilized in a first operation of the sequence of operations, and to display a second indication of a second different type of actuator movement of the plurality of different types of actuator movement, utilized in a second operation of the sequence of operations, following the first operation, as a result of movement of the actuator to perform the first operation of the sequence of operations.

In one aspect there is provided a method of using the delivery device, the method comprising: operating the actuator, when the indicator mechanism displays the first indication, in accordance with the first type of actuator movement, whereby as a result of operation of the actuator in accordance with the first type of actuator movement the indicator mechanism displays the second indication; operating the actuator, when the indicator mechanism displays the second indication, in accordance with the second different type of actuator movement.

In one aspect there is provided a method of manufacturing a delivery device, the method comprising: providing the delivery portion, the barrel portion, the plunger portion, and the indicator mechanism; and assembling the delivery portion, the barrel portion, the plunger portion, and the indicator mechanism to form the delivery device.

The plurality of different types of actuator movement may include at least one movement of the actuator in the axial direction relative to the barrel portion. The plurality of different types of actuator movement may include at least one rotation of the actuator about an axis parallel to the axial direction. The indicator mechanism may comprise a ring that is configured for rotation, from a first position in which the first indication is displayed, to a second position in which the second indication is displayed, as a result of the movement of the actuator to perform the first operation of the sequence of operations. The actuator may be configured for movement of the first type to perform a third operation of the sequence of operations, following the second operation, and the indicator mechanism may be configured to return to displaying the first indication as a result of movement of the actuator to perform the second operation of the sequence of operations.

### BRIEF DESCRIPTION OF THE FIGURES

Embodiments of the present disclosure will now be described, by way of example, with reference to the accompanying drawings in which:

FIG. 1 is an illustration showing a three-dimensional view of an exemplary delivery device in a locked state, with a delivery head in a non-delivery configuration;

FIG. 2 is an illustration showing a three-dimensional cross-sectional view of the delivery device of FIG. 1, through a transverse plane perpendicular to a longitudinal axis of the delivery device when in the locked state, with the delivery head in a non-delivery configuration;

FIG. 3 is an illustration showing a plunger tip element of the delivery device of FIG. 1;

FIG. 4A illustrates a plunger core of the delivery device of FIG. 1;

FIG. 4B is an illustration showing a cross-sectional view of the plunger core of FIG. 4A;

FIG. 5A is an illustration showing an actuator of the delivery device of FIG. 1 in more detail; and

FIG. 5B is an illustration showing a cross-sectional view of the actuator of FIG. 5A;

FIG. 6A is an illustration showing a plunger drive sleeve of the delivery device of FIG. 1;

FIG. 6B is an illustration showing a cross-sectional view of the plunger drive sleeve of FIG. 6A;

FIG. 7A is an illustration showing a guide element of the delivery device of FIG. 1;

FIG. 7B is an illustration showing the guide element of FIG. 7A with the plunger core of FIGS. 4A and 4B received in the guide element at a position corresponding to the locked state of the delivery device illustrated in FIG. 2;

FIG. 8A illustrates a three-dimensional view of a rotatable element of the delivery device of FIG. 1;

FIG. 8B is an illustration showing a side view of the rotatable element of FIG. 8A;

FIG. 8C is an illustration showing a cross-sectional view of the rotatable element of FIG. 8A;

FIG. 9A is an illustration showing a three-dimensional view of a locking mechanism for the delivery device of FIG. 1;

FIG. 9B is an illustration showing the locking mechanism of FIG. 9A, and the plunger core of FIGS. 4A and 4B, in which the plunger core is located at a position corresponding to the locked state of the delivery device;

FIG. 9C is an illustration showing the locking mechanism of FIG. 9A, and the plunger tip element of FIG. 3, in which the plunger tip element is located at a position corresponding to the locked state of the delivery device;

FIG. 10A is an illustration showing a three-dimensional view of an indicator ring for an indication mechanism of the delivery device of FIG. 1;

FIG. 10B is an illustration showing a cross-sectional view of the indicator of FIG. 10A;

FIG. 11A is an illustration showing a three-dimensional view of a locking ring for a decoupling, release and engage mechanism of the delivery device of FIG. 1;

FIG. 11B is an illustration showing a cross-sectional view of the locking ring of FIG. 11A;

FIG. 12 is an illustration showing, a three-dimensional view of the delivery device of FIG. 1 when in the locked state with the delivery head in a delivery-ready configuration;

FIG. 13 is an illustration showing a three-dimensional cross-sectional view of the delivery device in the configuration of FIG. 12, through a transverse plane perpendicular to a longitudinal axis of the delivery device;

FIG. 14 is an illustration showing, a three-dimensional view of the delivery device of FIG. 1 when in a pre-priming state;

FIG. 15 is an illustration showing a three-dimensional cross-sectional view of the delivery device in the configuration of FIG. 14, through a transverse plane perpendicular to a longitudinal axis of the delivery device;

FIG. 16 is an illustration showing, a three-dimensional view of the delivery device of FIG. 1 when in a primed-and-held state;

FIG. 17 is an illustration showing a three-dimensional cross-sectional view of the delivery device in the configuration of FIG. 16, through a transverse plane perpendicular to a longitudinal axis of the delivery device;

FIG. 18 is an illustration showing, a three-dimensional view of the delivery device of FIG. 1 when in a primed-and-released state;

FIG. 19 is an illustration showing a three-dimensional cross-sectional view of the delivery device in the configuration of FIG. 18, through a transverse plane perpendicular to a longitudinal axis of the delivery device;

FIG. 20 is an illustration showing, a three-dimensional view of the delivery device of FIG. 1 when in a post-delivery state; and

FIG. 21 is an illustration showing a three-dimensional cross-sectional view of the delivery device in the configuration of FIG. 20, through a transverse plane perpendicular to a longitudinal axis of the delivery device.

### DETAILED DESCRIPTION

### Overview

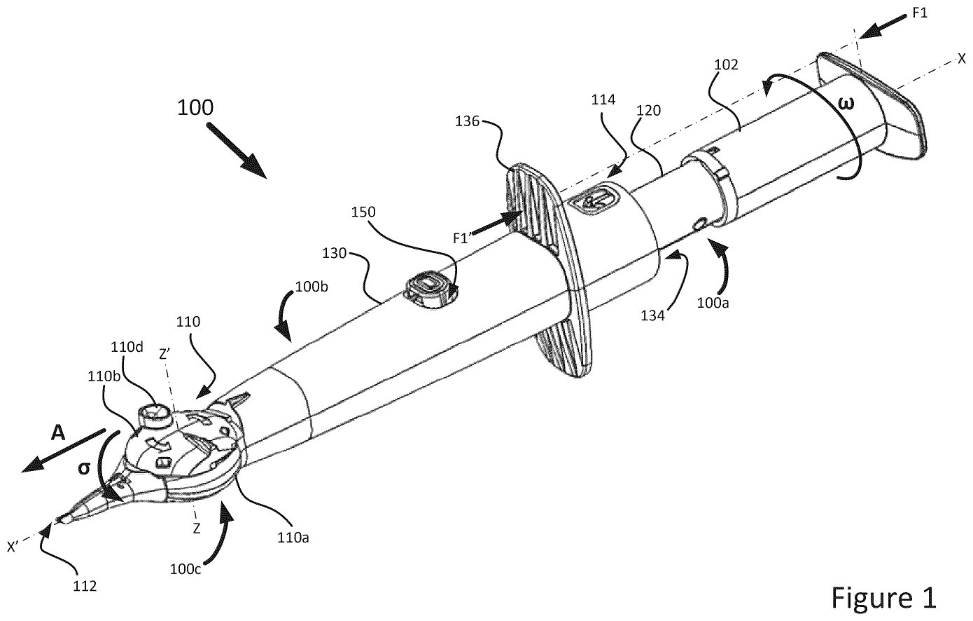

An exemplary delivery device for dispensing a viscous substance/intraocular lens (IOL), e.g., a viscous medium acting as a carrier for the IOL will now be described, in overview, by way of example only with reference to FIG. 1. Specifically, FIG. 1 is an illustration showing, schematically, a three-dimensional view of the exemplary delivery device generally at 100.

As seen in FIG. 1, the delivery device 100 resembles a medical syringe or the like. The delivery device 100 comprises a plunger portion 100a, a barrel portion 100b, and a delivery head 100c, which are respectively analogous to the plunger/piston, barrel, and nozzle/needle hub/needle of a conventional medical syringe.

The plunger portion 100a comprises the components of the delivery device 100 that work together allow a user to prime and operate the delivery device 100 to eject the viscous substance/IOL from the delivery device. These components include, at one end of the delivery device, an actuator 102 that is configured to be operated by a user, during delivery of the IOL, for example by application of a force by means of a thumb pressed on the actuator 102 in a similar manner to operation of a syringe.

The barrel portion 100b contains, when the delivery device 100 is assembled, part of the plunger portion 100a, while another part of the plunger portion 100a, including the actuator 102, extends from the barrel portion 100b to allow a user to interact with the actuator 102 to operate the delivery device 100.

The components of the plunger portion 100a and barrel portion 100b are mutually configured to allow movement of active components of the plunger portion 100a within, and relative to, barrel portion 100b when, during operation of the delivery device 100, a user applies a force between the actuator 102 and the barrel portion 100b (as indicated by arrows F1-F1′), for example by the user pressing the actuator 102 with a thumb while retaining the barrel portion 100b in position with two fingers.

The delivery head 100c comprises an IOL/OVD capsule 110 for containing the viscous substance/IOL, and a nozzle 112 for facilitating ejection of that viscous substance/IOL from the delivery head 100c as a result of the action of the plunger portion 100a. In the illustrated example, as described in more detail later, the delivery head 100c is configured for operation between a non-delivery configuration (as illustrated in FIG. 1) in which the IOL is contained within the delivery head 100c but the delivery head 100c is not ready for delivery, and a delivery-ready configuration in which the delivery head 100c is ready for delivery. Nevertheless, it will be appreciated that such operation is optional and the delivery head 100c may have a fixed configuration in which the delivery head 100c is always ready for delivery.

Beneficially, the internal features of the delivery device 100 are configured to decouple the force employed to deliver the IOL, from the force that is applied by a user. Specifically, as will be described in more detail later, the delivery device 100 is provided with an assisted drive mechanism configured such that, when a relatively low and constant force is applied to the actuator 102 by the user, the movement of the actuator 102 as it slides into the barrel portion 100b triggers corresponding movement of internal components of the plunger portion 100a that is driven entirely (or at least predominantly) by energy that is pre-stored within the delivery device (e.g., by a spring force exerted by a pre-compressed spring).

Beneficially, as will be described in more detail later, the internal components of delivery device 100 are configured to provide the assembled delivery device 100 with a number of distinct pre-delivery operational states, that a user operating the delivery device 100 steps through before a delivery operation can commence. These pre-delivery operational states include:
A ‘locked’, ‘safe’, ‘storage’, or ‘transit’, state;
A ‘pre-priming’ or ‘unlocked’ state;
A ‘primed-and-held’ state; and
A ‘primed-and-released’ state.

Specifically, prior to use, the device is in the locked state (the state illustrated in FIG. 1) in which the plunger portion 100a is inhibited from movement relative to the barrel portion 100b and so the delivery device 100 is effectively locked from use. In this state, the actuator 102 is extended at a maximal distance from the barrel portion 118b.

To facilitate this, the delivery device 100 is provided with a releasable locking mechanism 150 that is configured to retain the delivery device 100 in the locked state (and hence inhibited from use) until operated by a user to release the locking mechanism 150. The locking mechanism 150 provides a number of benefits including, for example, reducing or eliminating stress at the interfaces of the core mechanism during storage arising, for example, from the forces arising from the potential energy prestored in the delivery device 100. This therefore minimizes the risk of associated deterioration of the contact faces of various internal components, during storage, which might otherwise arise in the presence of a continuous contact pressure. The locking mechanism 150 also inhibits inadvertent movement of various components during storage or transit prior to use.

On release of the locking mechanism 150, the delivery device 100 enters the pre-priming state. In the pre-priming state of the delivery device 100, movement of the components of the plunger portion 100a, relative to the barrel portion 100b, under a force applied to the actuator 102, becomes possible. It will be appreciated that, in the illustrated example, as part of the procedure to configure the delivery device 100 into the pre-priming state, in addition to releasing the locking mechanism 150, the delivery head 100c may also be moved into the delivery-ready configuration. At this stage, the IOL is in a ‘pre-delivery’ position within the IOL/OVD capsule 110 of the delivery head 100c.

When the delivery device 100 is in the pre-priming state, the delivery device 100 can then be operated to enter the initial primed-and-held state. Specifically, the delivery device 100 is configured to enter the primed-and-held state as a result of linear movement of the components of the plunger portion 100a in a direction parallel to a longitudinal axis of the delivery device (e.g. in the direction of arrow A parallel to axis X-X′). At this stage the assisted drive mechanism is disengaged and, the linear movement is provided by a fully manual drive mechanism in which force applied by the user to the actuator 102, relative to the barrel portion 100b, to slide the actuator 102 towards (and into) the barrel portion 100b acts directly on a component of the plunger portion 100a, without assistance from the assisted drive mechanism, to push the IOL from its pre-delivery position within the IOL/OVD capsule 110 into a delivery-ready position within the nozzle 112.

Beneficially, the internal components of the delivery device 100 are configured to limit the extent to which the actuator 102 and associated components can move in the direction of arrow A to ensure that when the IOL is in the delivery-ready position within the nozzle 112, further movement of the IOL is inhibited while the delivery device 100 remains in the initial primed-and-held state. This hard stop provides the benefit that a user can safely push the actuator 102, until no further movement is possible, without risk of inadvertently pushing the IOL too far or accidentally ejecting the IOL all-together. When the limit of movement is reached the operator knows the delivery device 100 is appropriately primed, with the IOL in an optimum delivery position, without needing to look at the nozzle 112 to make a judgement on whether the end of the initial priming operation (the ‘dwell point’) has been reached. Accordingly, in the illustrated device, as the dwell point is predetermined, the need for operator judgement on the location of the IOL in the nozzle 112 is removed and the procedure is quicker, and easier to master, than would otherwise be the case. Moreover, having the assisted drive mechanism disengaged during this phase of the delivery procedure prevents accidental premature activation of the assisted drive mechanism (e.g., prior to the IOL being placed in the delivery-ready (primed) position in the nozzle 112).

When the delivery device 100 is in the primed-and-held state, the delivery device 100 can then be operated to enter the primed-and-released state. In the primed-and-released state, the manual drive mechanism is decoupled, the assisted drive mechanism is engaged, and the actuator 102 and associated components of the plunger portion 100a are released for linear movement in a direction parallel to a longitudinal axis of the delivery device (e.g. in the direction of arrow A parallel to axis X-X′). Specifically, the delivery device 100 is configured to enter the primed-and-released state as a result of, in the illustrated example, a quarter-turn (90°) rotational movement of the actuator 102 in the rotational direction indicated by the arrow ω. While a quarter-turn is provided as an example, it will be appreciated that any degree of rotation may be utilized. The actuator 102 is configured, as a result of this rotation, to engage with a decoupling, release and engage mechanism (referred to simply as the ‘decoupling mechanism’ for brevity when described in more detail later), to decouple the manual drive mechanism, and to engage and release the assisted drive mechanism.

Accordingly, when the delivery device 100 is in the primed-and-released state, the delivery device 100 is ready to deliver the IOL by application of a relatively low and constant force to the actuator 102. The resulting movement of the actuator 102, as it slides into the barrel portion 100b, triggers the assisted drive mechanism to provide assisted corresponding movement of the internal components of the plunger portion 100a, using the energy that is prestored within the delivery device, to press the IOL through the tightest cross-section of the nozzle 112, into the anterior chamber of the eye. The relatively high force used to achieve this (potentially up to 50N depending on lens selection and choice of viscoelastic agent) is provided by the prestored energy. When the force drops dramatically, once most of the lens has passed through the narrowest section of the nozzle 112, the fine control utilized to prevent trauma to the eye can still be provided by fine adjustment of the actuator 102.

The internal features of the delivery device 100 are, nevertheless, configured to ensure that, when the plunger portion 100a is in the primed-and-released state, and no force is applied to the actuator, the components of the plunger portion 100a are, nevertheless, maintained in position and the assisted drive mechanism will not move the internal components of the plunger portion 100a, using the energy that is prestored within the delivery device, without corresponding movement of the actuator 102 by the operator.

The various IOL delivery steps, or phases of operation, which are performed after the delivery device 100 is unlocked and in the pre-priming state, may be summarized as: push to prime, rotate to decouple, push to deliver.

Beneficially, as described in more detail later, the delivery device 100 is also provided with an indication mechanism configured to display a visual indication 114 of the next action that a user is to perform in the sequence of steps to deliver the IOL after the delivery device 100 has been unlocked, and is in the pre-priming state. In this example, the visual indication is in the form of an arrow indicating a direction in which the actuator 102 is to be moved by an operator, for example in a linear direction towards the nozzle 112 (as illustrated in FIG. 1) to enter the primed-and-held state, or in a rotational direction to enter the primed-and-released state. The provision of such an indication mechanism helps to make the delivery device more intuitive to use in a particularly elegant and effective manner.

Beneficially, the delivery device 100 is also provided with a resistance mechanism configured to provide an optimal amount of resistance to movement of the actuator 102, linearly in the direction of arrow A, by a user and to reduce the difference between static and kinetic friction. As described in more detail later, the resistance mechanism is configured to provide the resistance when the assisted drive mechanism is engaged, and the delivery device is being operated to deliver the IOL—i.e., while a delivery operation is being performed, after the delivery device 100 is placed in the primed-and-released state.

It can be seen, therefore, that the exemplary delivery device introduced above has application in an IOL delivery device (or similar device) for the safe and relatively effortless insertion of an IOL (or for the delivery of other objects or viscous materials), in a one-handed manner. In addition to lowering the force that a surgeon applies to force the lens through the nozzle and improving control when the lens exits the nozzle, the delivery device includes mechanisms that: increase the safety of the device; increase the longevity of the device, e.g., during prolonged storage; and that make the device easier to use and to learn to use.

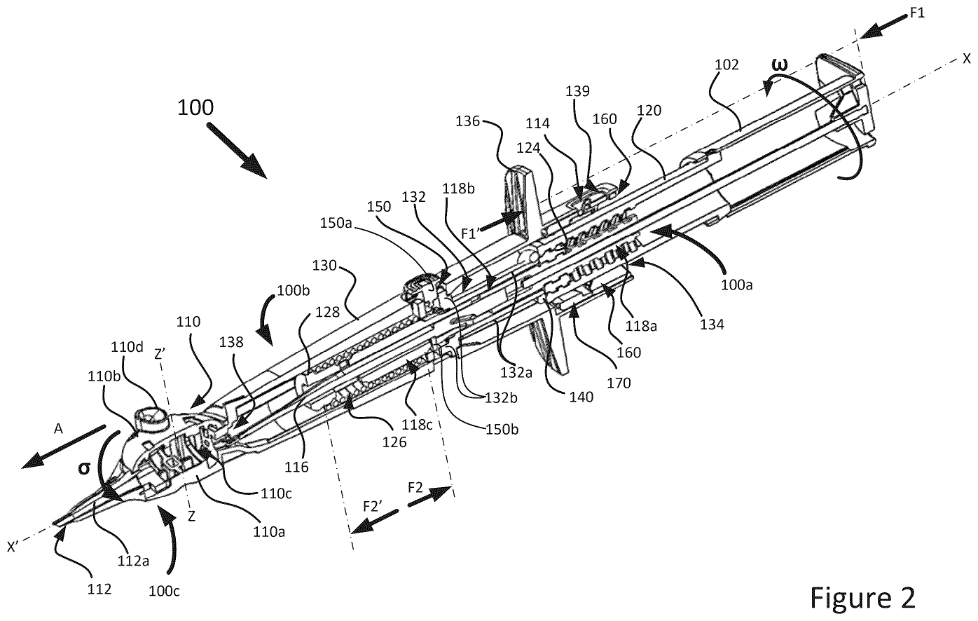

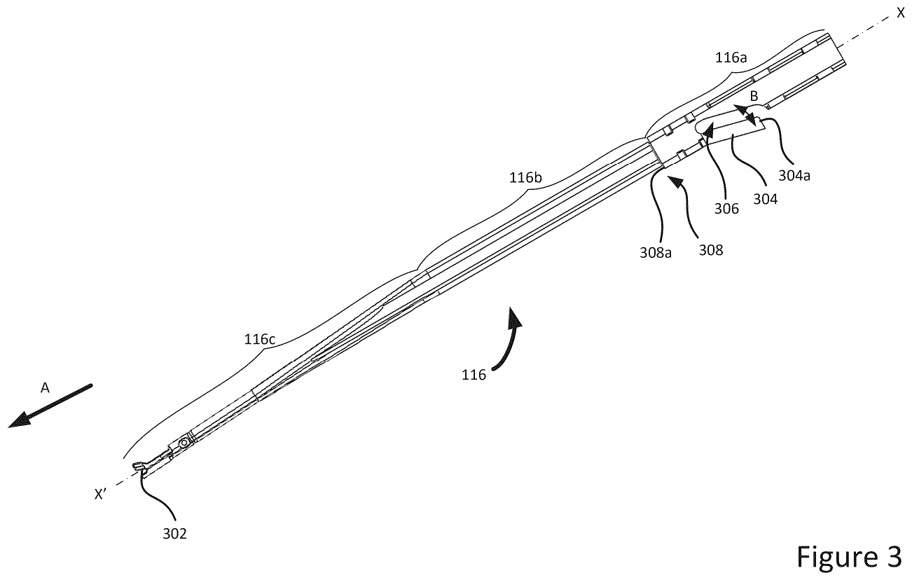

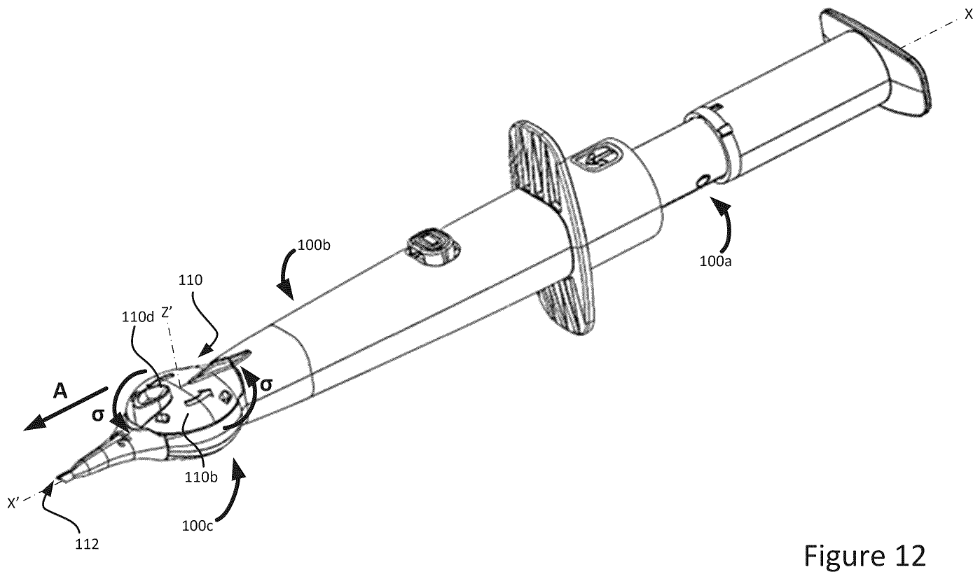

The general configuration of the main components and mechanisms of the delivery device 100 shown in FIG. 1 will now be described in broad terms with reference to FIGS. 1 and 2. A more detailed description of some of the components and mechanisms will then be provided with reference to FIGS. 3 to 11. Operational steps for delivery of an IOL using the delivery device 100 will then be described briefly with reference to FIGS. 12 to 21. Finally, possible material combinations that may, optionally, be used in fabrication of the delivery device will be described.

### General Configuration

As mentioned above, FIG. 1 shows the exemplary delivery device in a locked state with the delivery head 100c in a non-delivery configuration. FIG. 2 is an illustration showing, schematically, a three-dimensional cross-sectional view of the delivery device 100, through a transverse plane perpendicular to a longitudinal central axis of the delivery device 100, when in the locked state with the delivery head in a non-delivery configuration.

### Plunger Portion

As seen in FIG. 2, in addition to the actuator 102, the plunger portion 100a comprises a plunger tip element 116, a plunger core 118a, 118b, 118c (118), a plunger drive sleeve 120, a rotatable element 124, and a spring 126, that are arranged generally coaxially relative to the longitudinal axis of the delivery device (i.e. relative to axis X-X′), when the delivery device 100 is assembled. These elements will now be introduced briefly, with a more detailed description of each element provided later.

The plunger tip element 116 is an elongate element that is configured for applying, during operation, a force to the IOL to advance the IOL within the delivery head 100c and ultimately to eject the IOL through the nozzle 112.

The plunger core 118 is a generally elongate tubular element that, when the delivery device 100 is assembled, forms part of the assisted drive mechanism together with the rotatable element 124, and the spring 126. Specifically, the plunger core 118 is configured to act as a plunger driver that drives movement of, the plunger tip element 116, when the plunger core 118 and plunger tip element 116 are appropriately engaged with one another.

The plunger core 118 comprises, a threaded portion 118a, a central portion 118b, and a spring receiving portion 118c arranged along the central axis (X-X′). As seen in FIG. 2, when the delivery device 100 is assembled, the threaded portion 118a is located furthest from the nozzle 112, extends part of the length of the plunger core 118, and terminates at the first distal end of the plunger core 118. The spring receiving portion 118c is located closest to the nozzle 112, extends part of the length of the plunger core 118, and terminates at a second distal end of the plunger core 118. The central portion 118b is located between the threaded portion 118a and the spring receiving portion 118c.

The rotatable element 124 is a dual threaded ring shaped/annular cylindrical element having an internal threaded surface and an external threaded surface. The threaded portion 118a of the plunger core 118 has an external surface that is threaded for allowing the rotatable element 124 to be screwed onto the threaded portion 118a (i.e., by means of the internal surface of the rotatable element 124) when the delivery device 100 is assembled, in the manner that a bolt receives a nut. The threads at the interface between the threaded portion 118a of the plunger core 118 and the rotatable element 124, when assembled, are mutually configured so that the rotatable element 124 is back-drivable with respect to the plunger core 118.

The central portion 118b of the plunger core 118 is configured for engagement with an internal guide element 132 provided in the barrel portion 100b, to inhibit rotation of the plunger core 118 during operation, while allowing linear movement parallel to the central axis (e.g., in the direction of arrow A).

The spring receiving portion 118c of the plunger core 118 is configured to hold the spring 126, when the delivery device 100 is assembled, and for retaining the spring between a spring retainer 128 provided at a distal end of the spring receiving portion 118c and the internal guide element 132 of the barrel portion 100b.

The plunger core 118 and the plunger tip element 116 are mutually configured to allow the plunger tip element 116 to be slidably received in a central passage through the plunger core 118. The plunger core 118 and the plunger tip element 116 are also mutually configured such that the plunger tip element 116 is slidable from a first ‘retracted’ position (which corresponds to the delivery device 100 being either in the locked state, as seen in FIG. 2, or unlocked in the pre-priming state) to a second ‘primed’ position (which corresponds to the delivery device 100 being either in the primed-and-held state, or in the primed-and-released state).

The plunger drive sleeve 120 is an elongate, generally cylindrical, hollow element having a central passage that is configured for slidable reception of one longitudinal end of the drive sleeve 120 into the actuator 102, and for slidable reception of the internal guide element 132 of the barrel portion 100b into the other longitudinal end of the drive sleeve 120. Specifically, the plunger drive sleeve 120 and the actuator 102 are mutually configured to allow a first longitudinal end (the ‘actuator’ end) of the plunger drive sleeve 120 to be coupled to, and slidably received into, one end of the actuator 102. Similarly, the plunger drive sleeve 120 and the internal guide element 132 of the barrel portion 100b are mutually configured to allow a portion at one end of the internal guide element 132 of the barrel portion 100b to be slidably received into an opening at a second longitudinal end (the ‘nozzle’ end) of the plunger drive sleeve 120.

The plunger drive sleeve 120 has an internal threaded surface extending into the drive sleeve 120, from the opening at the nozzle end, along part of its length. This threaded surface is threaded to allow the rotatable element 124 to be screwed into the opening at the nozzle end (i.e., by means of the external surface of the rotatable element 124), when the delivery device 100 is assembled, in the manner that a boss nut can receive a bolt.

### Barrel Portion

Referring to FIGS. 1 and 2 the barrel portion 100b comprises an external cover 130, and the internal guide element 132 mentioned above (which may also be referred to as a ‘post’). These elements will now be introduced briefly, with a more detailed description of each element provided later.

The external cover 130 comprises an elongate sheath like element that is coupled, at one end, to the delivery head 100c. The barrel portion cover 130 forms an internal chamber having an opening 134, arranged at an end (the ‘actuator end’) opposite the end (the ‘nozzle end’) coupled to the delivery head 100c. The opening 134 is configured for partially receiving the plunger portion 100a, into the internal chamber formed by the barrel portion cover 130, when the delivery device 100 is assembled and during use as described in more detail later.

The barrel portion 100b is also provided with a finger flange 136 extending from the cover 130 in a plane orthogonal to the central longitudinal axis (X-X′). The finger flange 136 is arranged to allow a user to apply a force between the actuator 102 and the barrel portion 100b (as indicated by arrows F1-F1′), for example by the user pressing the actuator 102 with a thumb while retaining the barrel portion 100b in position by two fingers engaged with the finger flange 136, in similar manner to the way a syringe might be used.

The cover 130 and the internal guide element 132 are mutually configured to inhibit relative movement between the cover 130 and internal guide element 132, and hence maintain a fixed relationship between one another, when the delivery device 100 is assembled.

The internal guide element 132 comprises a generally hollow and cylindrical plunger guide portion 132a for guiding the plunger drive sleeve 120 during operation and a locking mechanism housing 132b for housing/guiding components of the locking mechanism 150. The plunger guide portion 132a is the part of the internal guide element 132 of the barrel portion 100b that is configured for slidable reception into the opening at the nozzle end of the plunger drive sleeve 120 as described above.

The internal guide element 132 has a central passage that extends the length of the internal guide element 132 and that is coaxially aligned with the longitudinal axis X-X′ when the delivery device 100 is assembled.

The internal guide element 132 is configured to receive the plunger core 118 into the central passage, and to allow slidable movement of the plunger core 118 relative to the internal guide element 132 during operation. As seen in FIG. 2, the plunger core 118 is longer than the internal guide element 132 (along the longitudinal axes (X-X′)).

Accordingly, when the delivery device 100 is assembled, prior to use (in the locked state, as seen in FIG. 2, or unlocked in the pre-priming state), the plunger core 118 extends all the way through and out from both longitudinal ends of the internal guide element 132.

The spring receiving portion 118c of the plunger core 118 extends from one longitudinal end of the internal guide element 132 (the end at which the locking mechanism housing portion 132b is located) towards nozzle 112 of the delivery device 100. When the delivery device 100 is assembled, the spring 126 is received on the spring receiving portion 118c with one end of the spring 126 engaged against a distal end of the internal guide element 132 (the end at which the locking mechanism housing portion 132b is located) and the other end of the spring 126 retained by the spring retainer 128 provided at the distal end of the spring receiving portion 118c, to retain the spring 126 in its pre-compressed state.

The central portion 118b of the plunger core 118 is located in the central passage of the internal guide element 132, and is configured to engage with features of the internal guide element 132 within the central passage, to inhibit rotation of the plunger core 118 during operation, while allowing linear movement parallel to the central axis (e.g., in the direction of arrow A) as described above.

The threaded portion 118a of the plunger core 118 extends from the other longitudinal end of the internal guide element 132 (corresponding to a distal end of the plunger guide portion 132a) into the central passage of the plunger drive sleeve 120 towards the actuator 102. The rotatable element 124 is fully threaded onto the threaded portion 118a (e.g., located adjacent the central portion 118b) and abuts the distal end of the plunger guide portion 132a. The rotatable element 124 is also threaded into the nozzle end of drive sleeve 120, to the extent that (in the illustrated configuration) a transverse surface of the rotatable element 124 (closest to the nozzle 112) and the nozzle end of the drive sleeve 120 are generally flush.

It can be seen, therefore, that when the delivery device is assembled, and in its locked state, the compressed spring exerts a decompression force (F2-F2′) on both the spring retainer 128, and the end of the internal guide element 132. This decompression force tends to urge the plunger core 118 in a direction towards the nozzle 112. However, when the delivery device 100 is in the locked state seen in FIG. 2, the plunger core 118 is inhibited from moving by the locking mechanism 150 (described in more detail later).

It will be appreciated that even when the delivery device 100 is unlocked the plunger core 118 is still inhibited from moving, by the rotatable element 124 engaging against the distal end of the distal end of the plunger guide portion 132a. However, the force urging the plunger core 118 towards the nozzle 112 also results in a back-driving force being exerted on the rotatable element 124 that tends to urge rotation of the rotatable element 124 in a direction that would cause the rotatable element 124 to move linearly, relative to the threaded portion 118a (towards its distal end), as a result of the induced screwing action. It will be understood that such rotation would also cause rotation of the rotatable element 124 within the threaded part of the drive sleeve 120 which would, in turn, cause the drive sleeve to move linearly towards the nozzle 112 (i.e., to slide over the plunger guide portion 132a), as a result of the induced screwing action. Accordingly, for such rotation to take place, the drive sleeve 120 would need to be free to move towards the nozzle 112. Until the assisted drive mechanism is engaged, however, the drive sleeve 120 is inhibited from moving towards the nozzle 112 and so the rotatable element 124 is also inhibited from rotating.

### Delivery Head

Referring to FIGS. 1 and 2, the IOL/OVD capsule 110 of the delivery head 100c has a base portion 110a and a cap portion 110b that define an internal chamber 110c for containing the IOL (and/or any viscoelastic agent/OVD) when in the pre-delivery position. The cap portion 110b is rotatable about a transverse axis (Z-Z′) that is perpendicular to the longitudinal axis (X-X′), as indicated by arrow σ for changing the configuration of the delivery head 100c from the non-delivery configuration (illustrated in FIGS. 1 and 2) to the delivery-ready configuration. The cap portion 110b includes a viscoelastic agent/OVD port 110d via which a viscoelastic agent/OVD can be introduced into the internal chamber of the IOL/OVD capsule 110. The cap portion 110b and base portion 110a are mutually configured such that rotation of the cap portion 110b prepares an IOL present in the internal chamber for delivery through the nozzle 112, for example by rolling the IOL into a configuration suitable for delivery via the nozzle 112 and/or by positioning the IOL for delivery.

The Internal chamber of the IOL/OVD capsule 110 and the internal chamber of the barrel portion 100b are interconnected by an opening 138 that is configured to allow an end of the plunger tip element 116, of the plunger portion 100a, to enter the internal chamber of the IOL/OVD capsule 110, from the internal chamber of the barrel portion 100b, during operation as described in more detail later.

The nozzle 112 is arranged at the delivery end of the delivery device 100 with an IOL/OVD conduit 112a that extends coaxially relative to the longitudinal axis (X-X′), from an opening in the nozzle 112 at the delivery end of the delivery device 100, to the internal chamber of the IOL/OVD capsule 110. The nozzle 112 and IOL/OVD conduit 112a are mutually configured to allow an IOL/OVD contained by the IOL/OVD capsule 110 to be urged along the IOL/OVD conduit 112a for delivery through the opening at the end of the nozzle 112. This may occur, for example, as a result of a force applied to the IOL/OVD by the end of the plunger tip element 116 as the plunger tip element 106 moves into, and through, the internal chamber of the IOL/OVD capsule 110 and the IOL/OVD conduit 112a during use of the delivery device as described in more detail later.

### Resistance Mechanism

As mentioned above, the delivery device 100 is also provided with a resistance mechanism to provide a preconfigured amount of resistance to movement of the drive sleeve 120 (and hence actuator 102), when the assisted drive mechanism is engaged and the delivery device is being operated to deliver the IOL (e.g., while a delivery operation is being performed, after the delivery device 100 is placed in the primed-and-released state).

In the example shown in FIG. 1, the resistance mechanism comprises a resistance element 140 in the form of an elastomer ring of a suitable material having a relatively high coefficient of friction and elasticity (e.g., rubber or the like). When the delivery device 100 is assembled, the resistive element 140 (e.g., ring) extends around an outer surface of the opening adjacent the distal end of the plunger guide element 132a. As seen in FIG. 1, the resistance element 140 is arranged to mutually engage with the nozzle end of the drive sleeve 120, when the delivery device 100 is assembled. The resistance element 140 is configured to provide a generally constant resistive force to the drive sleeve 120 being slid onto the plunger guide portion 132a, and hence to corresponding movement of the actuator 102, during operation to deliver the IOL.

While the use of the elastomer ring provides benefits in terms of simplicity and compactness, the resistance mechanism may comprise any suitable mechanism for providing the described amount of resistance to movement of the actuator 102. For example, in an alternative arrangement, the resistance mechanism could comprise a spring provided around the plunger guide element 132a that is configured to be compressed (and hence provide an opposing decompression force) to the drive sleeve 120 being slid onto the plunger guide portion 132a, and hence to corresponding movement of the actuator 102, during operation to deliver the IOL.

The use of a spring instead of (or in addition to) an elastomer ring has the potential to provide enhanced safety and/or reduced wastage by reducing the risk of the mechanism ‘creeping’ and slowly ejecting the IOL, for example if the device is left in the primed-and-released state for a prolonged period prior to the delivery operation commencing.

### Locking Mechanism

Referring to FIG. 2 the locking mechanism 150 comprises a push to release safety locking mechanism 150 that is configured for movement between a first ‘locked’ configuration in which the delivery device 100 is in the locked state (and hence inhibited from use) and a second ‘unlocked’ or ‘released’ configuration in which the delivery device 100 is in the unlocked state (and hence can be operated freely for delivery of the IOL or the like).

The locking mechanism 150 comprises a push button 150a and a movable locking element 150b that, when the delivery device 100 is assembled, are located (at least partially) in the locking mechanism housing 132b of the internal guide element 132. These elements will now be introduced briefly, with a more detailed description of each element provided later.

Specifically, the push button 150a and the locking mechanism housing 132b are mutually configured for slidable reception of the button 150a into the housing, when pushed, into the barrel portion 100b. The button is configured to extend, when the locking mechanism 150 is in the locked configuration (and hence the delivery device 100 is in its locked state), from the locking mechanism housing 132b through an aperture provided in the barrel portion cover 130, to provide an upper surface that is raised, relative to the barrel portion cover 130, and that can be pushed by a user of the delivery device 100.

The movable locking element 150b is coupled to the button 150a, and is provided inside the locking mechanism housing 132b, of the internal guide element 132, within the internal chamber formed by the barrel portion cover 130 when the delivery device 100 is assembled.

The movable locking element 150b, button 150a, and the locking mechanism housing 132b are mutually configured for allowing guided movement of the locking element 150b, when the button 150a is pushed into the barrel portion 100b by a user, from a first ‘locked’ position to a second ‘unlocked’ or ‘released’ position. The first ‘locked’ position corresponds to the locking mechanism 150 being in its locked configuration, whereas the second unlocked position corresponds to the locking mechanism 150 being in its unlocked configuration.

It will be appreciated that while a push button based (un) locking mechanism is described and has some benefits, an alternative could be used. For example, to mitigate the risk that internal components catch on the locking element 150b (e.g., as it slides through the locking element 150b), an alternative may be used. For example, a removable locking element could be used (e.g., in the manner of a ‘pull-tab’ or the like) whereby the locking element is entirely removed from the delivery device when unlocking is performed.

### Indication Mechanism

Referring to FIG. 2 the indication mechanism is shown generally at 160.

The indication mechanism 160 comprises a ring shaped/annular cylindrical ‘indicator’ element (the ‘indicator ring’) that, when the delivery device 100 is assembled, extends around an internal surface of the barrel portion cover 130, flush with (or near flush with) the opening 134 at the actuator end of the cover 130.

As described in more detail later, the indicator ring is configured to engage with the actuator 102, and to be rotated between a plurality of different indicator positions in each of which a different respective visual indicator 114 is displayed through an indicator aperture 139 provided in the barrel portion covering 130. The plurality of indicator positions include a first indicator position in which a first indication is displayed (in this example an arrow) that indicates that the next procedural step involves pushing the actuator 102 in the direction of arrow A. The plurality of indicators also include a second indicator position in which a second indication is displayed (in this example another arrow) that indicates that the next procedural step involves rotating the actuator 102 in the direction of arrow ω. In more detail, the indicator ring is configured for rotation (in a rotational direction opposite to that indicated by arrow ω), from the first position to the second position, when the delivery device 100 is in the unlocked state and an operator of the delivery device pushes the actuator 102 (e.g., with a thumb) linearly towards (and into) the barrel portion 100b (to engage and interact with the indicator ring). It will be appreciated that, as a result of the linear movement of the actuator 102, the delivery device 100 enters the primed-and-held state. The indicator ring is also configured for rotation (in the rotational direction indicated by arrow ω), from the second position back to the second position, when the delivery device 100 is in the primed-and-held state and an operator of the delivery device performs a rotational movement of the actuator 102 a quarter-turn (90°) in the rotational direction indicated by the arrow ω. It will be appreciated that, as a result of the rotation of the actuator 102, the delivery device 100 enters the primed-and-released state.

### Decoupling, Release and Engage Mechanism

Referring to FIG. 2 the decoupling mechanism is shown generally at 170.

The decoupling mechanism 170 comprises a ring shaped/annular cylindrical ‘decoupling’ or ‘locking’ element (the ‘locking ring’) that, when the delivery device 100 is assembled, extends around an internal surface of the barrel portion cover 130, near the opening 134 at the actuator end, between the finger flange 136 and the indication mechanism (and hence the associated visual indication 114).

As described in more detail later, the decoupling mechanism 170 is configured to engage with the actuator 102, and to be rotated between a plurality of different configurations by the actuator 102, during operation. The plurality of different configurations include a first ‘manual drive coupled’ configuration in which the actuator 102 is coupled for manually driving the plunger tip element 116. The plurality of different configurations also include a second ‘manual drive decoupled’/‘assisted drive coupled’ configuration in which the actuator 102 is coupled to the assisted drive mechanism for driving the plunger tip element 116 with the assistance of the pre-stored energy in the compressed spring 126. In more detail, the decoupling mechanism 170 is configured for rotation (together with the indicator mechanism 160), from the first configuration to the second configuration, when the delivery device 100 is in the primed-and-held state and an operator of the delivery device performs the quarter-turn (90°) rotational movement of the actuator 102 in the rotational direction indicated by the arrow ω. While a quarter-turn is identified, it will be appreciated that any angle is also contemplated. It will also be appreciated that, as a result of the rotation of the actuator 102, the delivery device 100 enters the primed-and-released state.

A more detailed description of some of the components and mechanisms will then be provided with reference to FIGS. 3 to 11.

### Plunger Tip Element

FIG. 3 illustrates the plunger tip element 116 of the delivery device 100 in more detail.

As seen in FIG. 3 the plunger tip element 116 is an elongate element having an interlocking portion 116a, a body 116b, and a tapered portion 116c.

When the delivery device 100 is assembled, the tapered portion 116c is positioned towards the delivery end of the delivery device 100 (closest to the nozzle 112), whereas the interlocking portion 116a is positioned towards the actuator end of the delivery device 100. The body 116b extends along the central axis (X-X′) from the interlocking portion 116a to the tapered portion 116c.

The tapered portion 116c has a relatively soft shaped tip 302, that is configured for applying, during operation, a force to the IOL to advance the IOL within the delivery head 100c and ultimately to eject the IOL through the nozzle 112.

The interlocking portion 116a of the plunger tip element 116 has a transverse cross-sectional shape that is configured for engaging with a corresponding transverse cross-sectional shape of the central passage of the plunger core 118, to allow the plunger tip element 116 to be slidably received into the central passage of the plunger core 118. The transverse cross-sectional shape of the interlocking portion 116a and corresponding transverse cross-sectional shape of the central passage of the plunger core 118 are also mutually configured to inhibit rotation of the plunger tip element 116 within, and relative to, the plunger core 118 during operation (while still allowing linear movement parallel to the central axis, e.g., in the direction of arrow A).

The interlocking portion 116a of the plunger tip element 116 is also provided with a resilient core engagement element 304 that extends from the interlocking portion 116a at an acute angle relative to the central longitudinal axis (X-X′), in a direction away from the tip 302, to present a protruding core engagement surface 304a (which may extend orthogonal, or near orthogonal, to the central longitudinal axis) that projects from the rest of the interlocking portion 116a. The core engagement element 304 by virtue of its resilience is configured to allow the core engagement element 304: to be flexed, when pressed, into an associated recess 306 that is provided in the interlocking portion 116a; and to spring out of the recess 306 when released (as indicated by arrows B). The recess 306 is configured such that when the resilient core engagement element 304 is pressed into the recess 306, the core engagement surface 304a no longer protrudes relative to the interlocking portion 116a.

The interlocking portion 116a and body 116b of the plunger tip element 116 are mutually configured to provide a step 308 in the cross-sectional profile of the plunger tip element 116 to provide a ‘locking’ surface 308a (which, in the illustrated example extends orthogonally, or near orthogonally, to the central axis (X-X′)) facing towards the nozzle 112. Specifically, the interlocking portion 116a of the plunger tip element 116 has a transverse cross-sectional width that is wider than a corresponding transverse cross-sectional width of the body 116b of the plunger tip element 116 to provide the step 308 and corresponding locking surface 308a. The locking surface 308a is configured to engage against a corresponding locking element 150b of the locking mechanism 150, when the delivery device 100 is in its locked state (shown in FIGS. 1 and 2), to inhibit movement of the plunger tip element 116 in a direction towards the nozzle 112 (i.e., along the longitudinal axis (X-X′) in the direction of arrow A).

Plunger Core

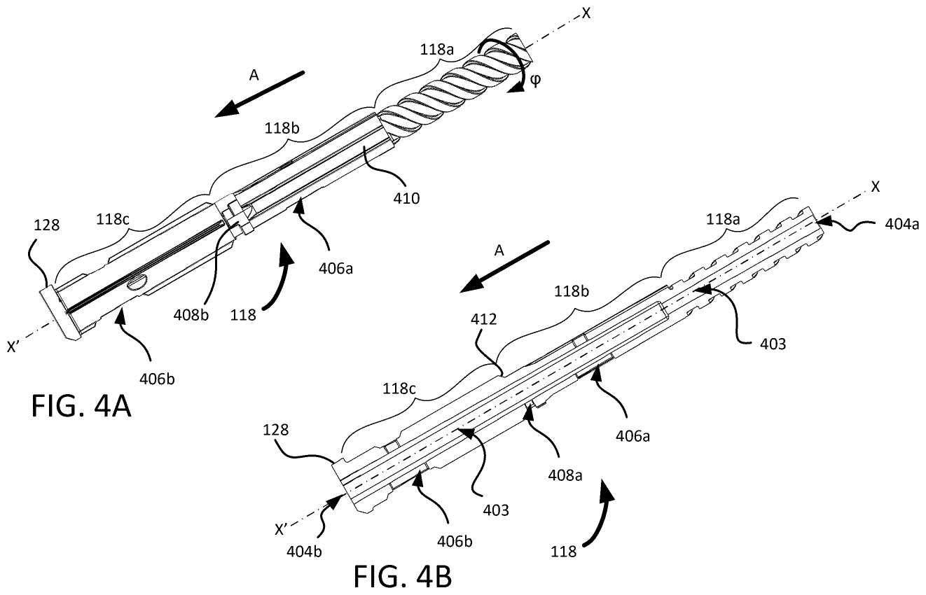

FIG. 4A illustrates the plunger core 118 of the delivery device 100 in more detail and FIG. 4B is an illustration showing a transverse cross-sectional view of the plunger core 118 along the central longitudinal axis (X-X′).

FIGS. 4A and 4B show, in particular, the arrangement of threaded portion 118a, central portion 118b, and spring receiving portion 118c arranged along the central axis (X-X′).

Referring to FIGS. 4A and 4B, the spring retainer 128 is generally annular, or part annular, flange that extends around the second end of the plunger core 118 (closest to the nozzle 112 when assembled).

The central passage of the plunger core 118 is shown at 403 in FIG. 4B. The central passage 403 extends the full length of the plunger core 118 between a first opening 404a at the first distal end of the plunger core 118 (in the threaded portion 118a) and a second opening 404b at the second end of the plunger core 118 (in the spring receiving portion 118c/spring retainer 128).

The central passage 403 is configured to slidably receive the interlocking portion 116a, the body 116b, and at least part of the tapered portion 116c of the plunger tip element 116. Specifically, the dimensions of the central passage 403 and the plunger tip element 116 are mutually configured to allow the interlocking portion 116a of plunger tip element 116 to be slidably received into the central passage 403 of the plunger core 118 via the second opening 404b at the second distal end. However, the central passage 403 and the core engagement element 304 of the plunger tip element 116 are mutually configured such that, when the interlocking portion 116a of the plunger tip element 116 is received into the central passage 403, the core engagement surface 304a of the core engagement element 304 would protrude beyond the outer perimeter of central passage 403 if the core engagement element 304 were in its unflexed natural state (i.e., not pressed into recess 306). Accordingly, for the interlocking portion 116a of the plunger tip element 116 to slide freely within the central passage 403 of the plunger core 118, the core engagement element 304 needs to be pressed at least partially into the corresponding recess 306 in the plunger tip element 116.

The central passage 403 of the plunger core 118 is provided with a plurality of core engagement element reception apertures (or recesses) 406a, 406b, in the wall defining the central passage 403, distributed along its length. The plurality of core engagement element reception apertures 406a, 406b are configured for receiving the core engagement element 304 of the plunger tip element 116 in an unflexed state, when the body 116b of the plunger tip element 116 is located at a corresponding position in the central passage 403. A first of the apertures 406a, located in the central portion 118b, is configured to receive the core engagement element 304 when the plunger tip element 116, is in the first retracted position (the position illustrated in FIG. 2). A second of the apertures 406b, located in the spring receiving portion 118b, is configured to receive the core engagement element 304 when the plunger tip element 116, is in the second primed position.

The plunger core 118 is also provided with at least one further ‘locking’ aperture (or recess) 408a, in the wall defining the central passage 403. The locking aperture/recess is configured for receiving part of the locking element 150b of the locking mechanism 150, when the locking element 150b is in the locked configuration (and the delivery device 100 is therefore in its locked state). A portion of the plunger core 118 surrounding and defining the locking aperture/recess is configured for engaging with the locking element 150b of the locking mechanism 150, when the locking element 150b is in the locked configuration, to inhibit movement of the plunger core 118 in a direction towards the nozzle 112 (i.e., along the longitudinal axis (X-X′) in the direction of arrow A).

The plunger core 118 is also provided with a pair of further locking projections 408b that extend from the central portion 118b configured for engaging with a surface of one or more corresponding features of the locking element 150b of the locking mechanism 150, when the locking element 150b is in the locked configuration, to inhibit movement of the plunger core 118 in a direction towards the nozzle 112 (i.e., along the longitudinal axis (X-X′) in the direction of arrow A).

As mentioned above, and as seen in FIGS. 4A and 4B, the threaded portion 118a of the plunger core 118 has an external surface that is threaded for allowing the rotatable element 124 to be screwed onto the threaded portion 118a. As seen in FIGS. 4A and 4B, the threaded surface is threaded in a manner that allows the rotatable element 124 to be screwed onto the threaded portion 118a by rotation in a rotational direction opposite to that indicated by arrow φ.

The central portion 118b of the plunger core 118 is of approximately the same length as the internal guide element 132 provided in the barrel portion 100b and, as seen in FIGS. 4A and 4B, comprises at least one keying feature 410 (in this example a plurality of such features). These keying features 410 are configured for engaging with mutually at least one mutually compatible keying feature, provided in the plunger guide portion 132a of the internal guide element 132, to inhibit rotation of the plunger core 118 when the central portion 118b of the plunger core 118 has been received in a central passage of the plunger guide portion 132a of the internal guide element 132. In this example, each keying feature 410 of the central portion 118b comprises a spline that extends the length of the central portion 118b. Correspondingly, each keying feature of the internal guide element 132 comprises a corresponding groove that extends along the central passage of the plunger guide portion 132a, for the length of the plunger guide portion 132a, and is configured to engage with the keying feature(s) (e.g., a corresponding spline) 410. It will be appreciated, however, that any suitable mechanism may be used to inhibit rotation of the plunger core 118.

The spring receiving portion 118c, and the central portion 118b of the plunger core 118, are mutually configured such that when the central portion 118b is slidably received into the central passage of the internal guide element 132, during assembly, the spring receiving portion 118c will, ultimately, engage against the distal end of the internal guide element 132 that is closest to the nozzle 112 (the end at which the locking mechanism housing 132b is located), and inhibit further movement of the central portion 118b into the central passage. To facilitate this, the spring receiving portion 118c, and the central portion 118b of the plunger core 118, are mutually configured to provide a step 412 for inhibiting the further movement of the central portion 118b into the central passage of the internal guide element 132. Specifically, the spring receiving portion 118c has a transverse cross-sectional width that is wider than a corresponding transverse cross-sectional width of the central portion 118b, and the central passage of the internal guide element 132.

The spring receiving portion 118c and spring 126 are mutually configured for providing a decompression force (F2-F2′) on both the spring retainer 128, and the end of the internal guide element 132 when the device is assembled before use. Specifically, the internal diameter of the spring 126 and external diameter/transverse width of the spring receiving portion 118c are mutually configured to allow the spring 126 to be received over the spring receiving portion 118c. The internal and externals diameters of the spring 126 are configured to ensure that the spring will be adequately retained between the spring retainer 128 and the internal guide element 132 of the barrel portion 118b. The length of the spring receiving portion 118c and characteristics of the spring (e.g., compression spring rate, compression spring pitch, number of coils, free length etc.) are mutually configured to provide the decompression force (F2-F2′) when the device is assembled before use.

### Actuator

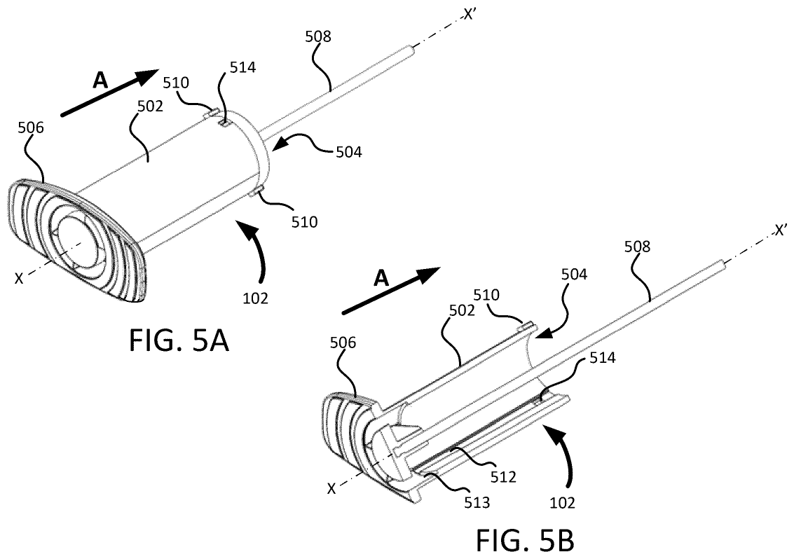

FIG. 5A illustrates the actuator 102 of the delivery device 100 in more detail and FIG. 5B is an illustration showing a cross-sectional view of the actuator 102.

The actuator 102 comprises a hollow, generally cylindrical, tubular body 502 that is provided with a circular opening 504 at one end, and a thumb flange or ‘plate’ 506 closing the tubular body 502 at the other. The actuator 102 also comprises a rod 508 that is mechanically coupled to the thumb flange 506, and is arranged to extend coaxially with the tubular body 502, along the central longitudinal axis (X-X′), towards the nozzle 112 of the delivery device 100 when assembled (e.g., as seen in FIG. 2).

The body 502 is provided with a plurality of projections or ‘tabs’ 510 (although it will be appreciated that there may be a single such projection/tab) distributed around a perimeter of the circular opening 504 and that extend from an external surface of the body 502 in a radial direction away from the central longitudinal axis (X-X′). These projections are configured to act as cams for interacting with and operating the indicator mechanism 160 and decoupling mechanism 170 as will be described in more detail later.

The actuator 102 and plunger drive sleeve 120 are mutually configured to allow the actuator 102 to slidably receive one end of the second plunger drive sleeve 120 into the opening 504 in the body 502 of the actuator 102. To facilitate slidable reception of the plunger drive sleeve 120 while inhibiting relative rotation between the actuator 102 and drive sleeve 120, the internal surface of actuator body 502 includes a plurality of guide channels 512 each extending parallel to the central axis (X-X′), from the opening 504 at one end of the actuator body 502, to a respective point near to the other end of the body 502 adjacent the thumb flange 506. Each guide channel 512 terminates at a circumferential channel 513 extending generally orthogonally to the guide channel 512 around part of the inner surface of the body 502 adjacent the thumb flange 506. To facilitate coupling with the plunger drive sleeve 120, the actuator body 502 is provided with a respective coupling aperture (or recess) 514 in each guide channel 512, near the perimeter of the circular opening 504.

These coupling apertures 514 and channels 512 are configured to interact with corresponding features of the plunger drive sleeve 120, to couple the actuator 102 and plunger drive sleeve 120 together, as will be described in more detail later.

The rod 508 and plunger core 118 are mutually configured such that the rod 508 is slidably receivable into the central passage 403 of the plunger core 118 via the opening 404a at the first distal end of the plunger core 118 (in the threaded portion 118a). Specifically, transverse cross-sectional width (e.g. diameter for a rod of circular cross-section as illustrated) the rod 508 is no greater than the minimum transverse cross-sectional width (e.g. diameter) of the plunger core 118.

The rod 508 has a length that is configured such that when the delivery device 100 is assembled, and in the locked state (i.e., with the actuator 102 at its maximal extension), the rod 508 extends to a location near but, in this example, slightly spaced from the interlocking portion 116a of the plunger tip element 116 (when the plunger tip element 116 is in the retracted position), e.g., as seen in FIG. 2. Specifically, the rod 508 is configured to extend from the thumb flange or ‘plate’ 506, through the tubular body 502 of the actuator 102, the drive sleeve 120, and the central passage 403 of the plunger core 118 to the location near but spaced from the interlocking portion 116a. It will be appreciated that while having the rod 508 spaced from the plunger tip element 116 is beneficial, the rod 508 could potentially be configured to touch the plunger tip element 116. The rod 508, the plunger core 118, and plunger tip element are also mutually configured such that when the delivery device is in the primed-and-held state the rod 508 engages against the actuator end of the plunger tip element 116.

The rod 508 forms part of the manual drive mechanism and is configured to engage with the plunger tip element 116, via the central passage 403 of the plunger core 118, to push the plunger tip element 116 from the retracted position to the primed position (e.g., when the actuator 102 is pushed in the direction of arrow A to change the state of the delivery device 100 from the pre-priming state to the primed-and-held) state.

### Plunger Drive Sleeve

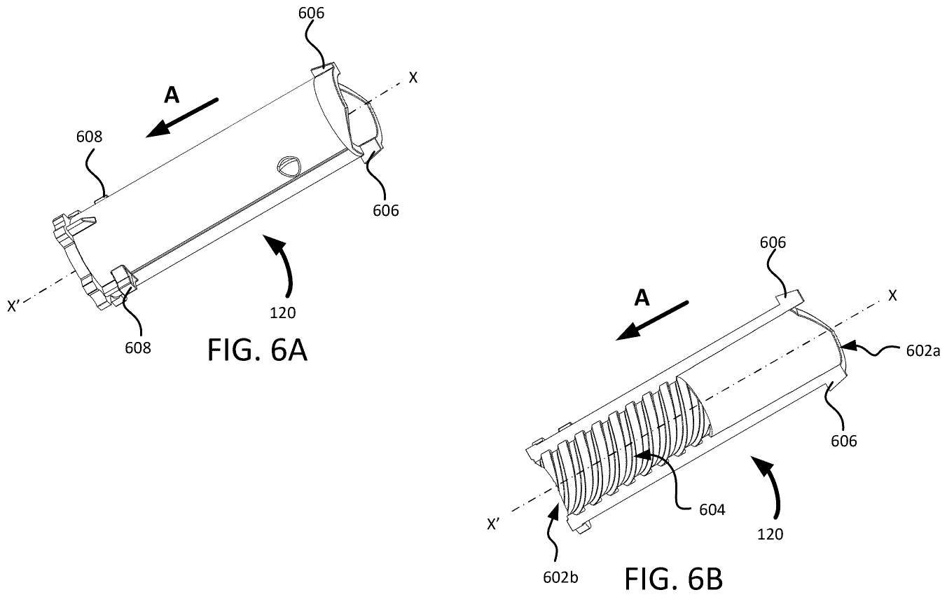

FIG. 6A illustrates the plunger drive sleeve 120 of the delivery device 100 in more detail and FIG. 6B is an illustration showing a cross-sectional view of the plunger drive sleeve 120.

As seen in FIGS. 6A and 6B, and as explained above, the plunger drive sleeve 120 comprises a hollow, generally cylindrical, tubular element of generally circular cross section with openings 602a, 602b at either end. The first opening 602a is located at an actuator end of the plunger drive sleeve 120 that is closest to the actuator 102, when the delivery device is assembled (e.g., as seen in FIG. 2). The second opening 602b is located at the nozzle end of the plunger drive sleeve 120 that is closest to the nozzle 112, when the delivery device is assembled (as seen in FIG. 2).

The threaded surface of the plunger drive sleeve 120 can be seen in FIG. 6B generally at 604. The threaded surface 604 is provided by means of one or more spiral grooves on the inside face of the plunger drive sleeve 120 to form the internal threaded surface 604. The threaded surface 604 extends from the second opening 602b towards the first opening for approximately half the length (in the illustrated example) of the drive sleeve 120 although the proportion of the sleeve used for the threaded surface may be different. This threaded surface 604 is configured for mutual engagement with the rotatable element 124 as it is screwed into the opening 602b at the nozzle end, when the delivery device 100 is assembled.

As indicated above, the actuator 102 and plunger drive sleeve 120 are mutually configured to allow the actuator 102 to slidably receive one end of the second plunger drive sleeve 120 into the opening 504 in the body 502 of the actuator 102. Specifically, the internal shape and dimensions of the actuator 102 and the external dimensions of the plunger drive sleeve 120 are configured to allow the actuator 102 to slide over the plunger drive sleeve 120 during operation.

To facilitate coupling with the actuator 102, the plunger drive sleeve 120 is provided with a plurality of coupling features 606 distributed around, and protruding from, an external surface of the drive sleeve 120 at the perimeter of the first opening. These coupling features 606 are configured to interact with corresponding coupling apertures or recesses 514 of the actuator 102, to couple the actuator 102 and plunger drive sleeve 120 together (e.g., as seen in FIG. 2). Specifically, the protruding coupling features 606 each have an angled surface that are configured to engage against the perimeter of the opening 504 at one end of the actuator body 502, during assembly to couple the drive sleeve 120 to the actuator 102 as the actuator end of the drive sleeve 120 is pushed into the opening 504, to cause a slight resilient flexing of the protruding coupling features 606 that allows the drive sleeve 120 to be slid into the actuator 102. Each protruding coupling feature 606 is also configured to engage in a respective guide channel 512 of the actuator 102 to allow axial movement, but to inhibit rotation of the drive sleeve 120, of the actuator 102 relative to the drive sleeve 120. Nevertheless, each protruding coupling feature 606 is also configured to engage in a respective circumferential channel 513 of the actuator 102, when the drive sleeve 120 is fully received into the actuator body 502 with end of the actuator 102 (e.g., a surface of the thumb flange 506 internal to the body 502) engaged against the actuator end of the drive sleeve 120. When the projection(s) are engaged in the circumferential channel(s) 513 some rotation of the actuator 102 relative to the drive sleeve 120 becomes possible.

The protruding coupling features 606 are also configured to engage with the coupling apertures/recesses, should a relative force be applied to pull the drive sleeve 120 from the actuator 102, in a manner that inhibits decoupling of the drive sleeve 120 from the actuator 102.

The drive sleeve 120 is also provided with a plurality of projections 608 (although it will be appreciated that there may be a single such projection) near to the second opening 602b at the nozzle end of the drive sleeve 12 and distributed around the external surface of the drive sleeve 120. Each projection 608 extends from the external surface of the drive sleeve 120 in a generally radial direction away from the central longitudinal axis (X-X′). These projections 608 are configured to engage with corresponding features of the locking ring of the decoupling mechanism 170 to inhibit movement of the drive sleeve 120 onto the plunger guide element 132a before the delivery device has been moved into the primed-and-released state as will be described in more detail later.

### Guide Element

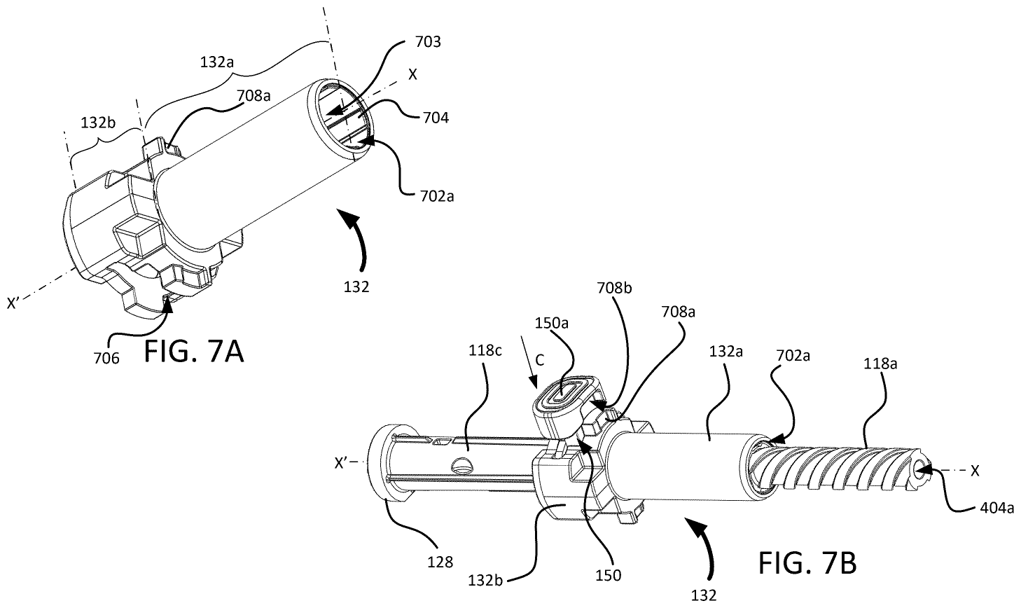

FIG. 7A is an illustration showing the internal guide element provided in the barrel portion 100b of the delivery device of FIG. 1 in more detail.

FIG. 7B is an illustration showing the internal guide element of FIG. 7A showing the plunger core of FIGS. 4A and 4B received in the internal guide element at a position corresponding to the locked state of the delivery device 100 as illustrated in FIG. 2. FIG. 7B also shows the button 150a of the locking mechanism 150 in position (in the locked configuration) but omits several features, e.g., including the spring and other features of the plunger portion for reasons of clarity.

FIGS. 7A and 7B show the internal guide element 132, and in particular, the generally hollow and cylindrical plunger guide portion 132a and locking mechanism housing 132b. The central passage of the internal guide element 132 is shown, generally at 703, and extends the length of the internal guide element 132 from an opening 702a in the distal end of the plunger guide portion 132a as described earlier.

As mentioned earlier, the central passage of the internal guide element 132 is provided with at least one keying feature, which is shown in FIG. 7A at 704. In the illustrated example, each keying feature comprises a corresponding groove that extends along the central passage from the opening, for the length of the central passage, and is configured to engage with the keying feature 410 of the central portion 118b of the plunger core 118 (as seen in FIG. 4A) to inhibit rotational movement of the plunger core 118 relative to the guide element 132 while allowing linear movement.

The locking mechanism housing 132b comprises a locking element guide 706 that extends through the locking mechanism housing 132b for receiving and guiding the locking element of the locking mechanism from the locked configuration to the unlocked configuration, during operation, when the button 150a is pressed (in the direction indicated by arrow C). The locking mechanism housing 132b also comprises one or more button engagement features 708a that are configured to engage with one or more corresponding housing engagement features 708b features when the button is pressed. The locking mechanism housing 132b and the button 150a are mutually configured to retain the button in the unlocked position when the button is pressed (e.g., as a result of friction between the button engagement feature(s) 708a of the locking mechanism housing 132b and corresponding housing engagement feature(s) 708b of the locking mechanism 150).

As seen in FIG. 7B, in the illustrated configuration, the plunger core 118 extends all the way through and out from both longitudinal ends of the internal guide element 132. The spring receiving portion 118c of the plunger core 118 extends from one longitudinal end of the internal guide element 132 (the end at which the locking mechanism housing portion 132b is located). The central portion 118b of the plunger core 118 is located in the central passage of the internal guide element 132 where it engages with the keying feature(s) of the internal guide element 132 within the central passage 703. The threaded portion 118a of the plunger core 118 extends from the other longitudinal end of the internal guide element 132 (corresponding to a distal end of the plunger guide portion 132a).

### Rotatable Element

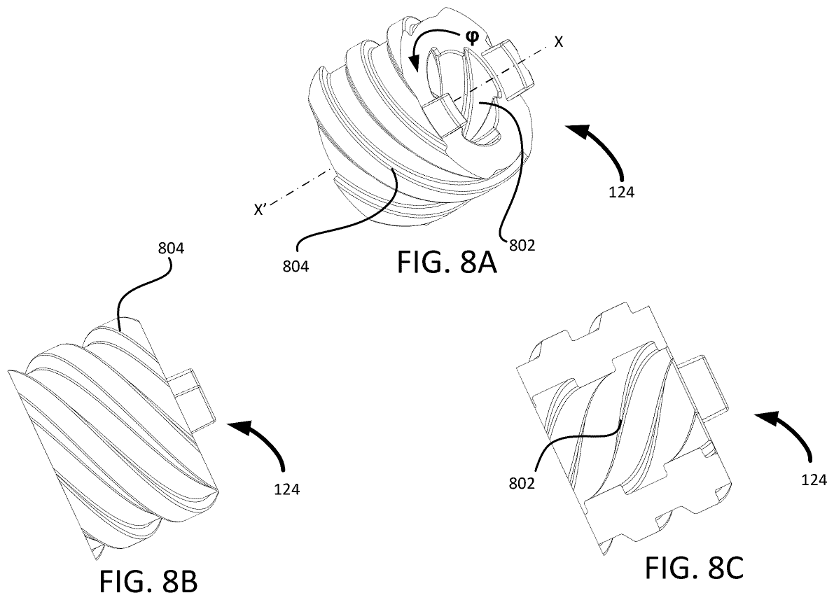

FIG. 8A illustrates a three-dimensional view of the rotatable element 124 of the delivery device 100. FIG. 8B is an illustration showing a side view of the rotatable element 124. FIG. 8C is an illustration showing a cross-sectional view of the rotatable element 124.

As seen in FIGS. 8A to 8C the rotatable element, in the exemplary delivery device of FIG. 1, is in the form of a nut. The nut 124 has an internal threaded surface 802 and an external threaded surface 804.

The internal threaded surface 802 of the rotatable element 124 and threaded surface of the threaded portion 118a of the plunger core 118 are mutually configured for engaging with one another to allow the rotatable element 124 to be threaded onto the threaded portion 118a, and to move linearly relative to the threaded portion 118a, as the rotatable element 124 rotates about the central axis (X-X′). In this example, the thread of the internal threaded surface 802 of the rotatable element 124 and the thread of the threaded portion 118a of the plunger core 118 are of relatively high pitch. Moreover the respective threads are mutually configured so that if the rotatable element 124 is rotated on the threaded portion 118a in a rotational direction, φ, relative to the central axis (X-X′), the displacement of the rotatable element 124 relative to the central portion 118b increases (and vice versa). The threads of the rotatable element 124 and threaded surface of the threaded portion 118a of the plunger core 118 are, in particular, mutually configured such that the rotatable element 124 is back-drivable with respect to the plunger core 118 (when assembled, e.g., as seen in FIG. 2) as a result of the force exerted by the spring 126.

In this example, the thread of the threaded portion 118a may, beneficially, have a plurality of thread starts although it will be appreciated that this need not be the case.

By way of illustration, in this example, a typical range for the pitch may be between once and twice the pitch diameter of the plunger thread although it will be appreciated that material selection is influential on the optimum pitch and so values outside this range may be suitable depending on the material used.

The external threaded surface 804 of the rotatable element 124 and the internal threaded surface 604 of the plunger drive sleeve 120 are mutually configured for engaging with one another to allow the rotatable element 124 to be threaded into the plunger drive sleeve 120, and to move linearly relative to the plunger drive sleeve 120, as the rotatable element 124 rotates about the central axis (X-X′). In this example, the thread of the external threaded surface 804 of the rotatable element 124 and the thread of the internal threaded surface 804 of the plunger drive sleeve 120 are of relatively high pitch and are mutually configured so that if the rotatable element 124 is rotated in the direction, q, relative to the central axis (X-X′) the displacement of the rotatable element 124 relative to the actuator end of the plunger drive sleeve 120 decreases (and vice versa). In this example, the thread of the internal threaded surface 604 of the plunger drive sleeve 120 may beneficially have a plurality of thread starts although it will be appreciated that this need not be the case.

### Locking Mechanism

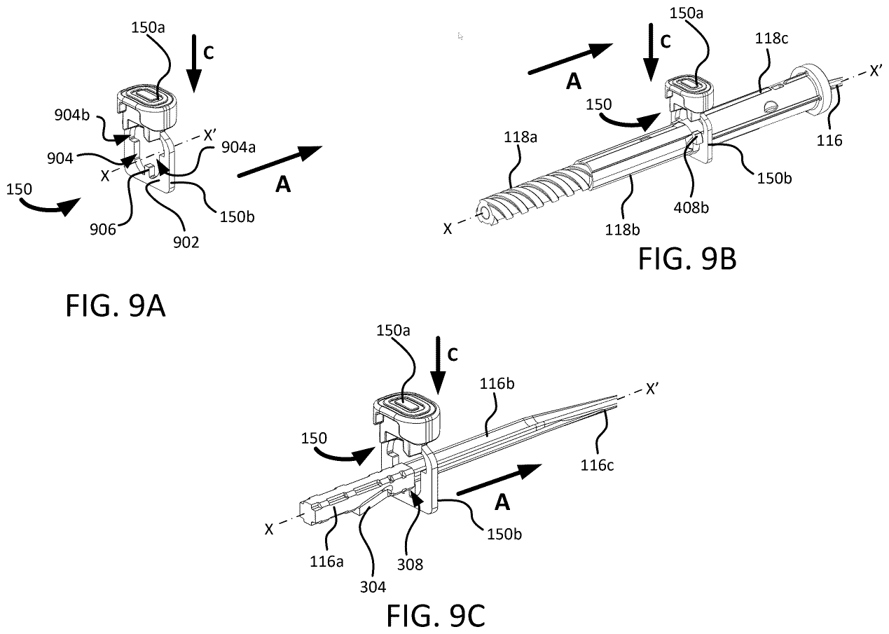

FIG. 9A illustrates a three-dimensional view of the locking mechanism 150 of the delivery device 100 in more detail. FIG. 9B is an illustration showing the locking mechanism of FIG. 9A and the plunger core 118 of FIGS. 4A and 4B in which the plunger core 118 is located at a position corresponding to the locked state of the delivery device 100 as illustrated in FIG. 2. FIG. 9C is an illustration showing the locking mechanism of FIG. 9A and the plunger tip element 116 of FIG. 3 in which the plunger tip element 116 is located at a position corresponding to the locked state of the delivery device 100 as illustrated in FIG. 2.

FIG. 9A shows, in particular the push button 150a and a movable locking element 150b of the locking mechanism 150.

As described above with reference to FIG. 2, the push button 150a is configured for slidable reception of the button 150a into the locking mechanism housing 132b and is configured to extend, when the locking mechanism 150 is in the locked configuration (and hence the delivery device 100 is in its locked state), from the locking mechanism housing 132b through an aperture provided in the barrel portion cover 130, to provide an upper surface that is raised, relative to the barrel portion cover 130, and that can be pushed by a user of the delivery device 100.

The movable locking element 150b is coupled to the button 150a and, in this example, is generally planer element (although other shapes are possible) that extends orthogonally relative to the central axis (X-X′) when the device is assembled. One planar surface of the locking element 150b (the surface that faces the actuator end when the delivery device is assembled) is configured to act as a locking surface 902 that engages with features of the plunger tip element 116 and the plunger core 118, when the locking mechanism 150 is in its locked configuration to inhibit movement of the plunger tip element 116 and the plunger core 118 in the direction of arrow A.

An aperture 904 is formed through the locking element 150b that is large enough to accommodate the plunger core 118 when coaxially aligned with the central axis (X-X′) of the delivery device 100 as shown in FIG. 9B. The shape of the aperture 904 is generally symmetrical (relative to a longitudinal plane extending in direction C through the central axis (X-X′)) and is configured to define a locking projection 906 that extends from the perimeter of the aperture in a direction opposite to the direction (indicated by arrow C) in which the button is pushed to move the locking mechanism into its unlocked configuration.

The locking projection 906 is configured to extend, when the locking mechanism 150 is in its locked configuration, through the locking aperture 408a provided in the wall defining the central passage 403 of the plunger core 118 described with reference to FIGS. 4A and 4B. As seen in FIG. 9C, the locking projection 906 is also configured to extend, when the locking mechanism 150 is in its locked configuration, such that the end of the locking projection 906 engages with the locking surface 308a provided by the step 308 between the interlocking portion 116a and body 116b of the plunger tip element 116 described with reference to FIG. 3. Hence, the locking surface 902 of the locking projection 906 will engage with the locking surface 308a of the plunger tip element 116 and the perimeter of the locking aperture 408a in the plunger core 118, when the locking mechanism 150 is in its locked configuration, to inhibit movement of the plunger tip element 116 and the plunger core 118 in the direction of arrow A.

The locking element 150b is configured such that the locking projection 906 will not extend into the locking aperture 408a, and will not engage with the locking surface 308a, when the locking mechanism 150 is in its unlocked configuration. Hence, the locking surface 902 of the locking projection 906 will not engage with the locking surface 308a of the plunger tip element 116 or the perimeter of the aperture 408a in the plunger core 118, when the locking mechanism 150 is in its unlocked configuration. Hence, movement of the plunger tip element 116 and the plunger core 118 in the direction of arrow A is not inhibited.

Moreover, the aperture 904 formed through the locking element 150b has a narrow part 904a and a wide part 904b. The narrow part 904a has a width that is large enough to accommodate the spring receiving portion 118c of the plunger core 118 when coaxially aligned with the central axis (X-X′) of the delivery device 100 as shown in FIG. 9B. The aperture 904 is configured such that, when the delivery device 100 is assembled, and locking mechanism 150 is in its locked configuration, the widest part of the plunger core 118 is aligned with the narrow part 904a. The aperture 904 is also configured such that, when the delivery device 100 is assembled, and locking mechanism 150 is in its unlocked configuration, the widest part of the plunger core 118 is aligned with the wider part 904b.

As seen in FIG. 4B, the narrow part 904a is, however, too narrow to allow the pair of further locking projections 408b that extend from the central portion 118b of the plunger core 118 (described with reference to FIGS. 4A and 4B) to pass through the narrow portion 904a in the direction of arrow A. In contrast the wide part 904b is sufficiently wide to allow the pair of further locking projections 408b that extend from the central portion 118b of the plunger core 118 (described with reference to FIGS. 4A and 4B) to pass through the aperture 904 in the locking element 150b, in the direction of arrow A. Hence, the locking surface 902 of the locking element adjacent the narrow part 904a will engage with the locking projections 408b of the plunger core 118, when the locking mechanism 150 is in its locked configuration, to inhibit movement of the plunger core 118 in the direction of arrow A. However, the wide part 904b will allow the locking projections 408b of the plunger core 118 to pass through the aperture 904, when the locking mechanism 150 is in its unlocked configuration, to allow movement of the plunger core 118 in the direction of arrow A.

### Indication Mechanism

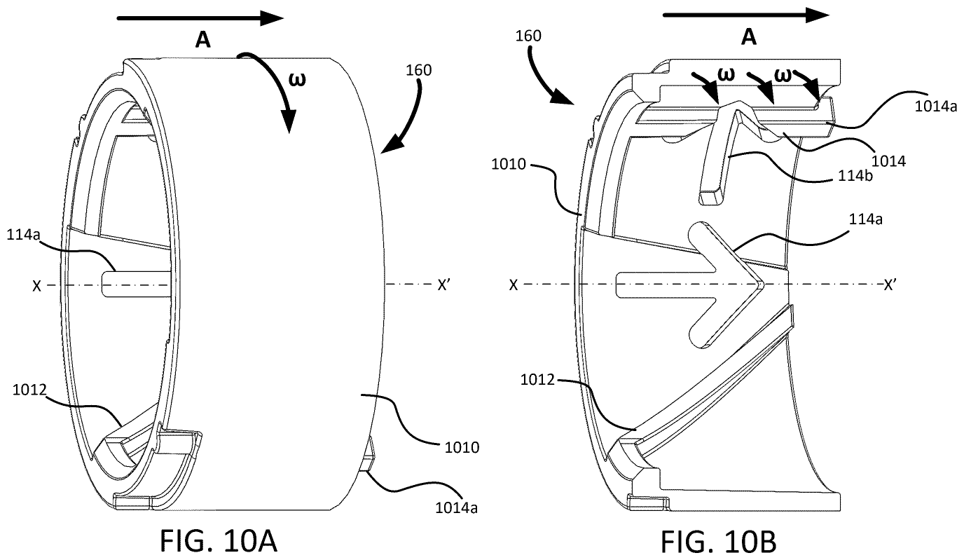

FIG. 10A is an illustration showing a three-dimensional view of the indicator ring for the indication mechanism 160 of the delivery device 100 in more detail.

FIG. 10B is an illustration showing a cross-sectional view of the indicator ring of FIG. 10A.

As described above, the indication mechanism 160 comprises a ring shaped/annular cylindrical ‘indicator’ element (the ‘indicator ring’). The indicator ring is shown in FIGS. 10A and 10B at 1010.

The indicator ring 1010 is configured for coaxial alignment with, and rotation around, the central axis (X-X′) when the delivery device 100 is assembled.

The indicator ring 1010 comprises a plurality of cam surfaces 1012 (although there may be a single such surface) and a plurality of visual indications 114 (114a and 114b).

As seen in FIG. 10B, the indicator ring 1010 includes a first visual indication 114a in the form of an arrow pointing in the direction of arrow A, and a second visual indication 114b in the form of an arrow pointing in the rotational direction of arrows ω. The visual indications 114 each comprise an aperture in the shape of the respective visual indication that extends through to the external surface of the indicator ring 1010 such that when the visual indication 114 is aligned with the indicator aperture 139 provided in the barrel portion cover 130 (e.g., as seen in FIG. 2) the visual indication 114 can be seen through the indicator aperture 139 provided in the barrel portion cover 130.

Referring to FIG. 10B, each cam surface 1012 is configured for respectively engaging with a corresponding projection 510 extending radially from an external surface of the body 502 of the actuator 102 (as described with reference to FIGS. 5A and 5B) when, in operation, the actuator 102 is pushed, in the direction of arrow A, into the opening 134 at the actuator end of the cover 130. The cam surface(s) 1012 and projection(s) 510 are mutually configured such that as the projections 510 move in the direction of arrow A, the projections apply a force to the cam surface(s) 1012 of the indicator ring 1010 to rotate the indicator ring 1010 in the direction opposite that indicated by the arrows ω.

Specifically, each cam surface 1012 is configured for respectively engaging with a corresponding projection 510 to rotate the indicator ring 1010 between different indicator positions in each of which a different respective visual indicator 114 is displayed through the indicator aperture 139 provided in the barrel portion covering 130.

The different indicator positions include a first indicator position in which the first visual indication 114a is displayed to indicate that the next procedural step involves pushing the actuator 102 in the direction of arrow A. It is this position that the indicator ring 1010 is in when the delivery device 100 is in its initial locked state prior to delivery. The different indicator positions also include a second indicator position in which the second visual indication 114b is displayed to indicate that the next procedural step involves rotating the actuator 102 in the direction of arrow ω. It is this position that the indicator ring 1010 moves to as a result of the engagement of each cam surface 1012 with corresponding projection 510 to rotate the indicator ring 1010.

Accordingly, in operation, the indicator ring 1010 rotates (in a rotational direction opposite to that indicated by arrow ω), from the first indicator position to the second indicator position, when the delivery device 100 is in the unlocked (pre-priming) state and an operator of the delivery device pushes the actuator 102 (e.g., with a thumb) linearly towards (and into) the barrel portion 100b (to engage and interact with the indicator ring 1010). Thus the visual indication 114 displayed in the through the indicator aperture 139 provided in the barrel portion covering 130 changes from the first visual indication 114a to the second visual indication 114b.

As seen in FIG. 10B, the indicator ring 1010 also comprises a plurality of ridges 1014 (although there may be a single such ridge) that extend axially along an internal surface of the indicator ring 1010.

Each ridge 1014 is also configured for respectively interacting with a corresponding projection 510 of the actuator 102. However, each ridge 1014 is configured to engage with the corresponding projection 510 when the indictor ring 1010 has already been rotated to its second indicator position and, in operation, the actuator 102 is subsequently rotated, in the rotational direction of arrows ω (e.g., to change the state of the delivery device 100 from the primed-and-held state to the primed-and-released state). Specifically, the ridge(s) 1014 and projection(s) 510 are mutually configured such that as the projections are rotated with the actuator 102 in the rotational direction of arrows w, the projections 510 move to engage with and then apply a force to the ridge(s) 1014 of the indicator ring 1010 to rotate the indicator ring 1010 in the direction of arrows ω.

Accordingly, in operation, the indicator ring 1010 rotates (in a rotational direction indicated by arrows ω), from the second indicator position back to the first indicator position, when the delivery device 100 is in the primed-and-held state and an operator of the delivery device rotates the actuator 102, in a rotational direction indicated by arrows ω, to change the state of the delivery device 100 to the primed-and-released state. Thus the visual indication 114 displayed through the indicator aperture 139 provided in the barrel portion covering 130 changes from the second visual indication 114b back to the first visual indication 114a.

Each ridge 1014 also projects slightly beyond a rim of the cylindrical wall forming the indicator ring 1010 in the direction of arrow A to form a projecting portion 1014a. Specifically, each projecting portion 1014a extends beyond the rim of the cylindrical wall forming the indicator ring 1010 that interfaces with a corresponding rim of the locking ring of the decoupling mechanism 170 when the delivery device 100 is assembled. Each projecting portion 1014a is configured to engage in a corresponding indicator ring engagement recess provided in a cylindrical wall forming the locking ring, when the delivery device 100 is assembled, as described in more detail later.

### Decoupling, Release and Engage Mechanism (‘Decoupling Mechanism’)

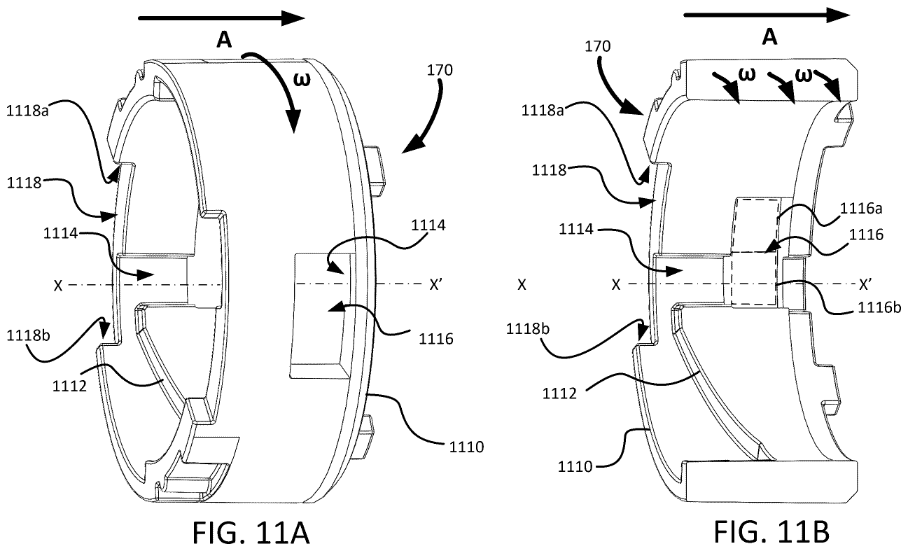

FIG. 11A is an illustration showing a three-dimensional view of the locking ring for the decoupling, release and engage mechanism 170 of the delivery device 100 in more detail.

FIG. 11B is an illustration showing a cross-sectional view of the locking ring of FIG. 11A.

As described above, the decoupling mechanism 170 comprises a ring shaped/annular cylindrical ‘decoupling’ or ‘locking’ element (the ‘locking ring’). The locking ring is shown in FIGS. 11A and 11B at 1110.

The locking ring 1110 is configured for coaxial alignment with, and rotation around, the central axis (X-X′) when the delivery device 100 is assembled. When the delivery device 100 is assembled, before the delivery device 100 is in the primed-and-released state, the locking ring 1110 is in the first manual drive coupled configuration described above with reference to FIG. 2.

The locking ring 1110 comprises, on/in its internal surface, a plurality of cam surfaces 1112 (although there may be a single such surface), a plurality of guide channels 1114 (although there may be a single such channel), and a plurality of locking apertures 1116 (although there may be a single such aperture).

The locking ring 1110 also comprises a plurality of indicator ring engagement recesses 1118 (although there may be a single such recess) of the cylindrical wall forming the locking ring 1110. Specifically, as seen in FIGS. 11A and 11B each indicator ring engagement recess 1118 extends into the rim of the cylindrical wall forming the locking ring 1110 that interfaces with a corresponding rim of the indicator ring 1010 of the indicator mechanism when the delivery device 100 is assembled. Each indicator ring engagement recess 1118 extends around a portion of the circumference of the locking ring 1110 and is configured for receiving a corresponding projecting portion 1014a of a ridge 1014 of the indicator ring 1010 described with reference to FIGS. 10A and 10B, when the device is assembled.

Each indicator ring engagement recess 1118 and the corresponding ridge 1014 and projecting portion 1014a are mutually configured such that the projecting portion 1014a will move from one end of the recess to the other when the indicator ring 1010 is rotated from its first indicator position to its second as described with reference to FIGS. 10A and 10B. Specifically, each indicator ring engagement recess 1118 is configured such that, when the indicator ring 1010 is in its first indicator position (shown in FIGS. 10A and 10B, the corresponding projecting portion 1014a is located at a first end 1118a of that recess 1118 in the rotational direction w. Each indicator ring engagement recess 1118 is also configured such that, when the indicator ring 1010 is in its second indicator position (shown in FIGS. 10A and 10B), the corresponding projecting portion 1014a is located at a second end 1118b of that recess 1118 in a direction opposite to the rotational direction ω.

Each cam surface 1112 is configured for initially engaging with a corresponding projection 510 extending radially from an external surface of the body 502 of the actuator 102 (as described with reference to FIGS. 5A and 5B) when the delivery device is in the primed-and-held state and the indicator ring 1010 is in the second indicator position. Specifically each cam surface 1112 is configured to engage with a corresponding projection 510 to inhibit further movement of the actuator 102 in the direction of arrow A. The cam surface(s) 1112 and projection(s) 510 are, nevertheless, mutually configured such that as the actuator 102 is rotated in the rotational direction indicated by the arrows w to return the indicator ring 1010 to the first indicator position from the second indicator position the projection(s) 510 initially engage against the cam surface(s) 1112 of the locking ring 1110. The cam surface(s) 1112 are configured initially to guide the projection(s) 510, as the actuator 102 begins to rotate, to force the actuator 102 to move a short distance in a direction opposite to that indicated by arrow A.

The reverse movement of actuator 102 causes a corresponding movement of the rod 508 of the actuator 102 (described with reference to FIGS. 5A and 5B to disengage the rod 508 from the plunger tip element 116 and hence decouple the manual drive mechanism (after the plunger tip element 116 has been moved from its retracted position to its primed position). The reverse movement of actuator 102, in combination with the rotational movement in the rotational direction indicated by the arrows w, also brings the projection(s) 510 on the actuator 102 into engagement with the projecting portion 1014a of the ridge(s) 1014 to begin to induce the corresponding rotation of the indicator ring 1010 ultimately to return the indicator ring 1010 to the first indicator position.

Each guide channel 1114 extends axially the width of the locking ring 1110 and is configured for allowing a corresponding projection 510 of the actuator 102 to move in the direction of arrow A, guided by the channel, when that projection 510 is aligned with that guide channel 1114 and the actuator 102 is pushed in the direction of arrow A.

The cam surface(s) 1112 and projection(s) 510 of the actuator 102 are also mutually configured such that the rotation of the actuator 102, in the rotational direction indicated by the arrows ω, also moves the projection(s) 510 into axial alignment with the corresponding guide channel 1114 of the locking ring 1110. This axial alignment occurs shortly after the projection(s) 510 move into engagement with the projecting ridge(s) 1014 of the indicator ring 1010 before the indicator ring 1010 has fully returned to the first indicator position. The guide channel(s) 1114 and projection(s) 510 of the actuator 102 are mutually configured such that following this axial alignment with the guide channel(s) 1114, further rotation of the actuator 102 in the rotational direction indicated by the arrows ω (to continue to move the indicator ring 1010 back to the first indicator position) also induces associated rotation of the locking ring 1110 in the rotational direction indicated by the arrows ω. Specifically, guide channel(s) 1114 and projection(s) 510 are mutually configured such that further rotation of the actuator 102 causes each projection 510 to respectively engage against a wall of a corresponding guide channel 1114 to induce the associated rotation of the locking ring 1110.

Each locking aperture 1116 comprises a generally rectangular hole through the cylindrical wall defining the locking ring 1110, near the rim of the locking ring that is closest to the nozzle 112 of the assembled delivery device 100. The longest dimension of each locking aperture 1116 respectively extends, generally orthogonally from a corresponding guide channel 1114, in the rotational direction of arrows ω. Each guide channel 1114 crosses a respective locking aperture 1116. Thus, each locking aperture has a first part 1116a that is not aligned with the corresponding guide channel 1114 and a second part 1116b that is aligned with the with the corresponding guide channel 1114.

Each locking aperture 1116 is respectively configured to receive a corresponding projection 608 that extends from the external surface of the drive sleeve 120 (described with reference to FIGS. 6A and 6B). Each locking aperture 1116 is respectively configured such that the corresponding projection 608 of the drive sleeve 120 extends into the first part 1116a of that locking aperture 1116, when the locking ring 1110 is in the first manual drive coupled configuration, before the delivery device 100 enters the primed-and-released state. The locking aperture 1116 and projections 608 are mutually configured such that when a projection 608 of the drive sleeve 120 extends into the first part 1116a of a corresponding locking aperture 1116, that projection 608 engages with a perimeter of the first part 1116a of the corresponding locking aperture 1116 to inhibit movement of the drive sleeve 120 onto the plunger guide element 132a before the delivery device has been moved into the primed-and-released state.

The locking aperture(s) 1116 are further configured such that, when the actuator 102 is subject to the further rotation that induces associated rotation of the locking ring 1110, the rotation of the locking ring 1110 causes the locking aperture(s) 1116 to move to a position in which the projection(s) 608 of the drive sleeve 120 are aligned with the guide channel(s) 1114. Thus, when the projection(s) 608 of the drive sleeve 120 are aligned with the guide channel(s) 1114, movement of the drive sleeve 120, onto the plunger guide element 132a, is no longer inhibited.

It will be appreciated that the locking ring 1110 may also be configured such that, when the locking ring moves into a position in which the projection(s) 608 of the drive sleeve 120 are aligned with the guide channel(s) 1114, the guide channel(s) 1114 move into alignment with axial guide channels provided within the barrel portion cover 130 for guiding the drive sleeve 120 in the direction of arrow A while inhibiting relative rotational movement of the drive sleeve 120 relative to the barrel portion 100b.

The operational steps for delivery of an IOL using the delivery device 100 will be described with reference to FIGS. 12 to 21.

### Delivery Device Operation

### Delivery Head Configuration

As described earlier, FIGS. 1 and 2 show the delivery device in the locked state.

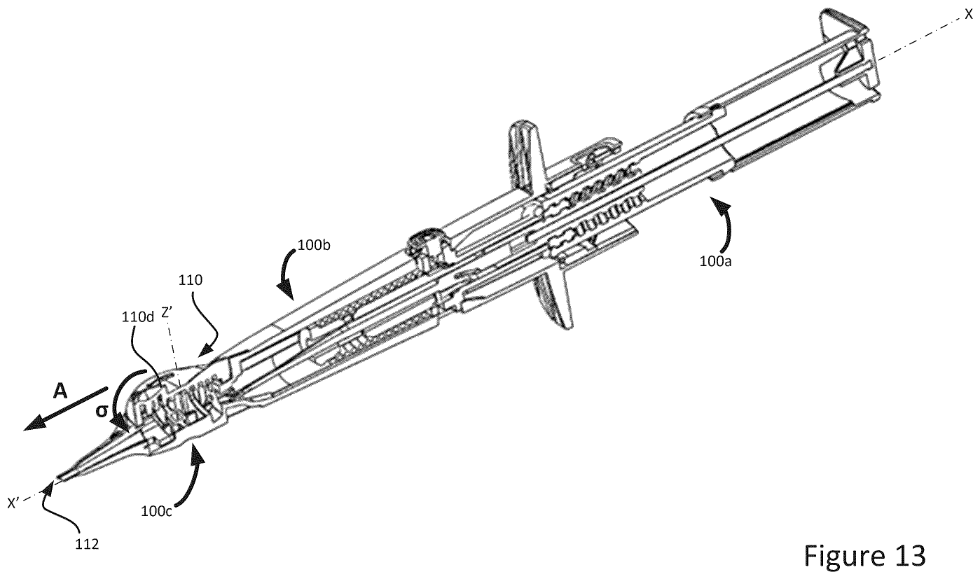

FIG. 12 is an illustration showing, a three-dimensional view of the delivery device 100 in the locked state following configuration of the delivery head 100c into the delivery-ready configuration. FIG. 13 is an illustration showing a three-dimensional cross-sectional view of the delivery device in the configuration of FIG. 12, through a transverse plane perpendicular to a longitudinal axis of the delivery device.

Referring to FIGS. 12 and 13, to change the configuration of the delivery head 100c of the delivery device 100 from the non-delivery configuration, to the delivery-ready configuration, the cap portion 110b of the delivery head 100c is rotated about the transverse axis (Z-Z′) perpendicular to the longitudinal axis (X-X′), as indicated by arrow σ, for a quarter turn (90°). Before or after this action is performed a viscoelastic agent/OVD may be introduced into the IOL/OVD capsule 110 via the viscoelastic agent/OVD port 110d.

In this example, the IOL is already present in the internal chamber of the IOL/OVD capsule 110 and the action of rotating the cap portion 110b of the delivery head 100c prepares the IOL for delivery through the nozzle 112.

Nevertheless, it will be appreciated that a delivery head of this type is optional and the delivery head 100c may have a fixed configuration in which the delivery head 100c is ready for delivery and the IOL is preprepared for delivery through the nozzle 112.

As seen in FIGS. 12 and 13, following the action to change the configuration of the delivery head 100c from the non-delivery configuration to the delivery-ready configuration, other than the changes to the delivery head, the various internal components of the delivery device 100 remain in essentially the same configuration as shown in FIGS. 1 and 2. Thus, the delivery device 100 shown in FIGS. 12 and 13 is still in the locked state.

### Locked State to Pre-Priming (Unlocked) State

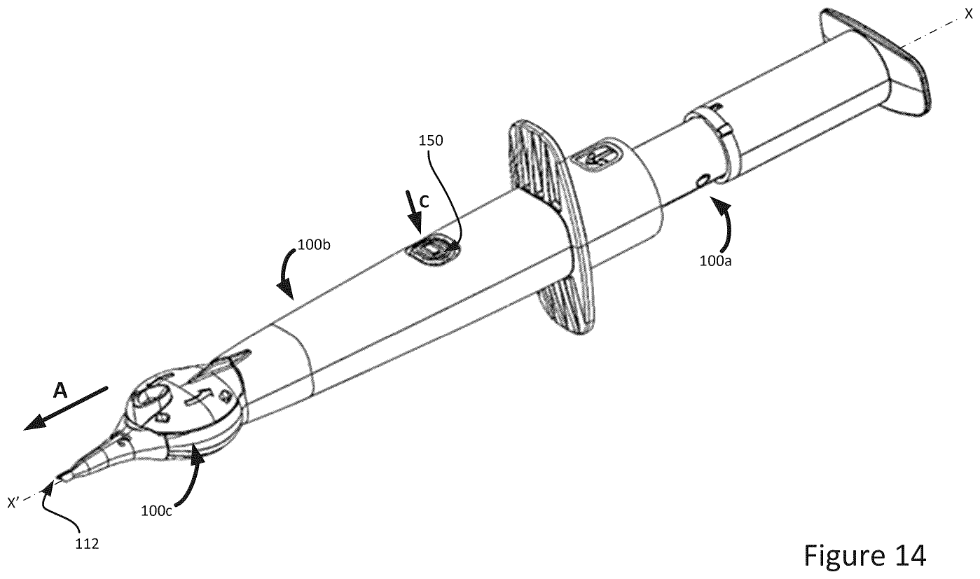

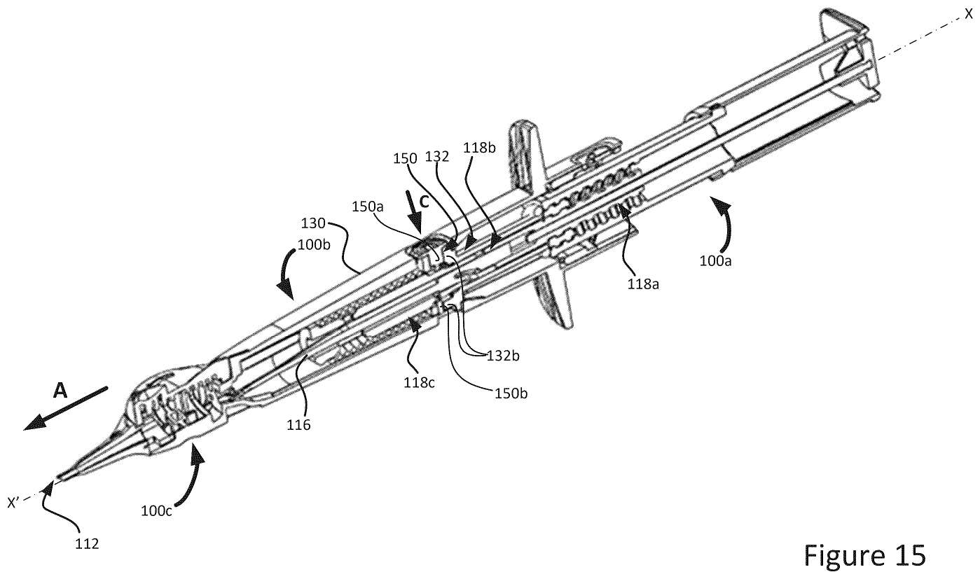

FIG. 14 is an illustration showing, a three-dimensional view of the delivery device 100 following an operation to change the state of the delivery device 100, from the locked state shown in FIGS. 12 and 13, to in the pre-priming (unlocked) state. FIG. 15 is an illustration showing a three-dimensional cross-sectional view of the delivery device in the configuration of FIG. 14, through a transverse plane perpendicular to a longitudinal axis of the delivery device.

Referring to FIGS. 14 and 15, to change the state of the delivery device 100 from the locked state, to the pre-priming (unlocked) state, the button 150a of the locking mechanism 150 is pressed by the user in the direction indicated by arrow C. This pushing action results in the button 150a sliding into the locking mechanism housing 132b until the button engagement feature(s) 708a of the locking mechanism housing 132b engage with the corresponding housing engagement features 708b feature(s) (as described with reference to FIGS. 7A and 7B). As seen in FIGS. 14 and 15, following the action to push the button 150a, an upper (exposed) surface of the button 150a is substantially flush with the barrel portion cover 130.

The movable locking element 150b that is coupled to the button 150a also moves, as a result of the pushing action, in the direction of arrow C and thus the locking projection 906 of the locking mechanism 150 (described with reference to FIGS. 9A to 9C) disengages from the plunger tip element 116 and moves out of the locking aperture 408a in the plunger core 118. Thus movement of the plunger tip element 116 and the plunger core 118 in the direction of arrow A is no longer inhibited by the projection 906 of the locking mechanism 150. The movement of the locking element 150b also aligns the wide part 904b of the aperture through the locking element 150b (described with reference to FIGS. 9A to 9C) appropriately with the pair of further locking projections 408b that extend from the central portion 118b of the plunger core 118 (described with reference to FIGS. 4A and 4B). Accordingly, the locking element 150b is moved into a position in which the plunger core 118 can move in the direction of arrow A, without being inhibited by the locking element 150b.

Referring to FIGS. 14 and 15, therefore, the action to push the button 150a, changes the state of the delivery device 100, from the locked state to the pre-priming (unlocked) state. Specifically, in the locked state the linear movement of plunger core 118 and the plunger tip element 116 is inhibited by the locking element 150b, whereas in the pre-priming (unlocked) state linear movement of plunger core 118 and the plunger tip element 116 is no longer inhibited by the locking element 150b.

Other than the changes to the locking mechanism 150, the various internal components of the delivery device 100 remain in essentially the same configuration as shown in FIGS. 12 and 13.

### Pre-Priming (Unlocked) State to Primed-and-Held State

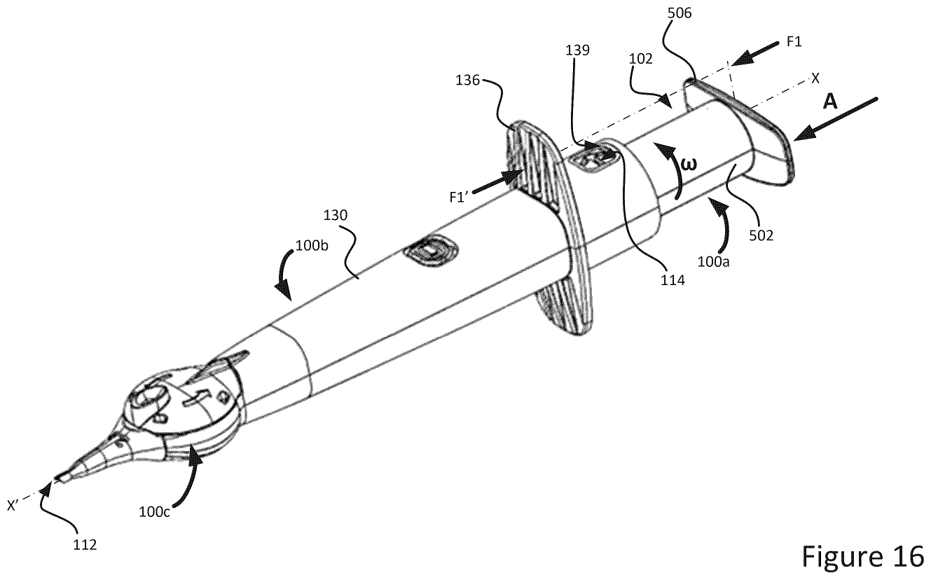

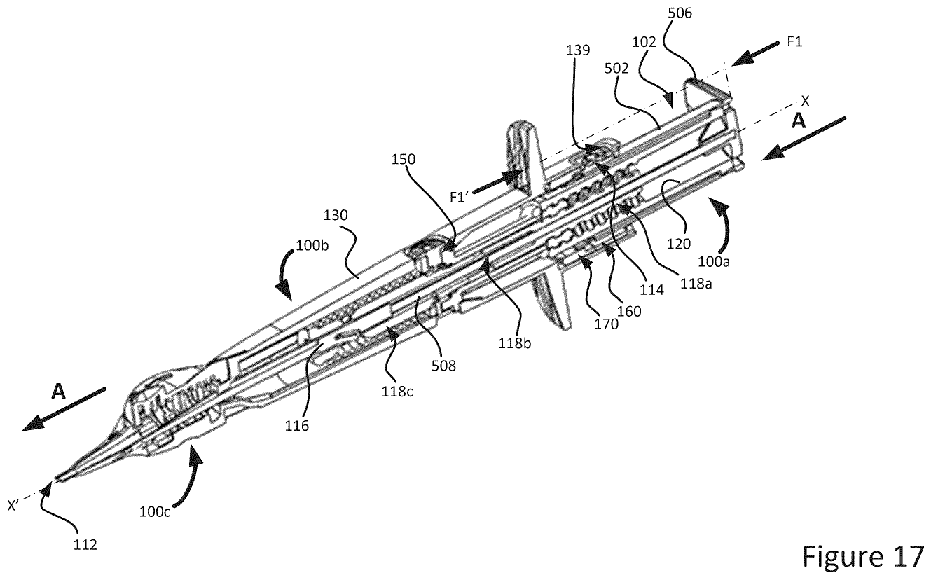

FIG. 16 is an illustration showing, a three-dimensional view of the delivery device 100 following an operation to change the state of the delivery device 100, from the pre-priming (unlocked) state of FIGS. 14 and 15, to the primed-and-held state. FIG. 17 is an illustration showing a three-dimensional cross-sectional view of the delivery device in the configuration of FIG. 16, through a transverse plane perpendicular to a longitudinal axis of the delivery device.

Referring to FIGS. 16 and 17, to change the state of the delivery device 100 from the pre-priming (unlocked) state to the primed-and-held state, the actuator 102 is pushed by a user in the direction of arrow A. Specifically, a user applies a force between the actuator 102 and the barrel portion 100b (as indicated by arrows F1-F1′), for example by the user pressing the actuator 102 with a thumb while retaining the finger flange 136 of the barrel portion 100b in position with two fingers.

As a result of the action to push the actuator 102, the actuator 102 slides over the plunger drive sleeve 120 until the projection(s) 510 of the actuator 102 (described with reference to FIGS. 5A and 5B) engage against corresponding cam surface(s) 1112 of the decoupling mechanism 170. During the action to push the actuator 102 the rod 508 of the actuator engages against the end of the plunger tip element 116 that is closest to the actuator. As the plunger tip element 116 is no longer inhibited by the locking mechanism 150, on engagement with the plunger tip element 116 the rod 508 (and hence the actuator 102) can continue to move, pushing the plunger tip element 116 in the direction of arrow A, from its retracted position towards the nozzle 112. It will be appreciated that, as the plunger tip element 116 moves within the central passage through the plunger core 118, the resilient core engagement element 304 of the plunger tip element 116 flexes into the corresponding recess 306 (as described with reference to FIG. 3). As the projection(s) 510 of the actuator 102 reach the corresponding cam surface(s) 1112 of the decoupling mechanism 170, the plunger tip element 116 is pushed into its primed position and the resilient core engagement element 304 of the plunger tip element 116 springs back from the recess 306 into the corresponding core engagement element reception aperture 406b of the plunger core 118 (described with reference to FIGS. 4A and 4B). Thus, the plunger tip element 116 becomes coupled to the plunger core 118.

Moreover, as the actuator 102 slides over the drive sleeve 120, before the projection(s) 510 of the actuator 102 reach the corresponding cam surface(s) 1112 of the decoupling mechanism 170, the actuator 102 engages with the indicator ring 1010 of the indicator mechanism 160 to move the indicator ring from the first indicator position to the second indicator position (as described with reference to FIGS. 10A and 10B). Thus, the visual indication 114 displayed through the aperture 139 provided in the barrel portion cover 130 changes from an arrow pointing in the direction of arrow A (as seen in FIG. 14) to an arrow pointing in the rotational direction of arrow ω (as seen in FIG. 16).

When the projection(s) 510 of the actuator 102 reach the corresponding cam surface(s) 1112 of the decoupling mechanism 170, further movement of the actuator 102, in the direction of arrow A, is inhibited by the locking ring 1110 of the decoupling mechanism 170 (described with reference to FIGS. 11A and 11B). Specifically, the projection(s) 510 on the actuator 102 (described with reference to FIGS. 5A and 5B) engage against the corresponding cam surface(s) 1112 and are thus inhibited from further movement in the direction of arrow A.

Moreover, when the end of the actuator 102 reaches the actuator end of the drive sleeve 120, the protruding coupling feature(s) 606 on the drive sleeve 120 engage in the circumferential channel(s) 513 of the actuator 102 and thus some rotation of the actuator 102 relative to the drive sleeve 120 becomes possible (as described with reference to FIGS. 6A and 6B).

Referring to FIGS. 16 and 17, therefore, the action to push the actuator 102 onto the drive sleeve 120 moves the plunger tip element 116 into a primed position and changes the state of the delivery device 100, from the pre-priming state to the primed-and-held state. Specifically, in the primed-and-held state plunger tip element 116 is primed but further linear movement of the actuator 102 is inhibited by the locking ring 1110 of the decoupling mechanism 170.

Other than the changes to the position of the actuator 102, plunger tip element 116, and indicator ring 1010 of the indicator mechanism 160, the various internal components of the delivery device 100 remain in essentially the same configuration as shown in FIGS. 14 and 15.

### Primed-and-Held State to Primed-and-Released State

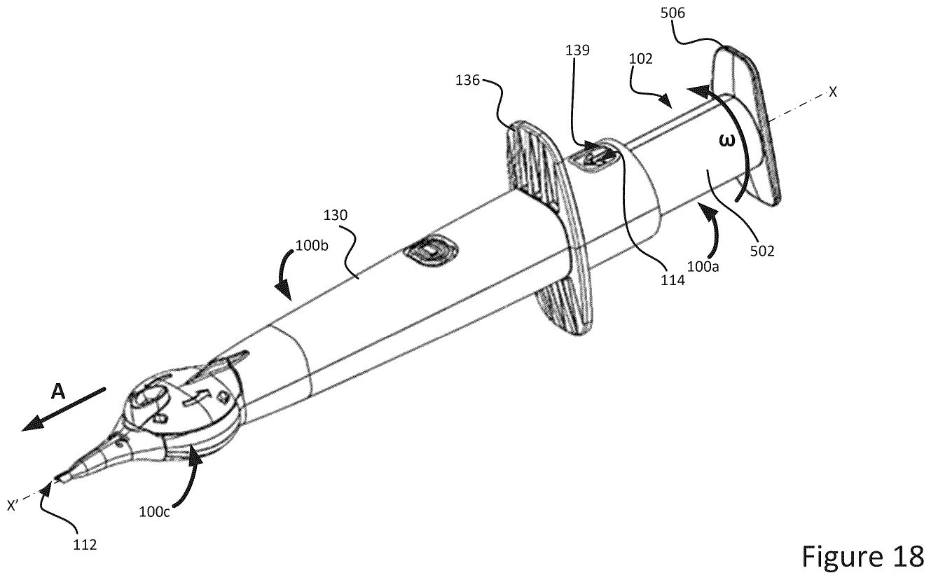

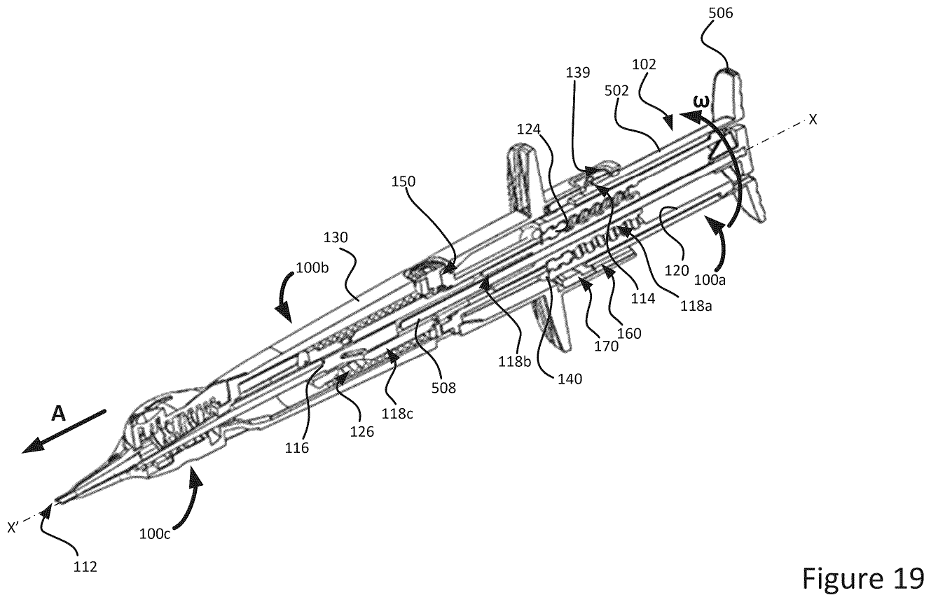

FIG. 18 is an illustration showing, a three-dimensional view of the delivery device 100 following an operation to change the state of the delivery device 100, from the primed-and-held state shown in FIGS. 16 and 17, to the primed-and-released state. FIG. 19 is an illustration showing a three-dimensional cross-sectional view of the delivery device in the configuration of FIG. 18, through a transverse plane perpendicular to a longitudinal axis of the delivery device.

Referring to FIGS. 18 and 19, to change the state of the delivery device 100 from the primed-and-held state to the primed-and-released state, the actuator 102 is rotated by a quarter turn (90°) in the rotational direction of arrow ω.

As a result of the action to rotate the actuator 102, the actuator 102 rotates relative to the plunger drive sleeve 120. As the actuator 102 rotates, the actuator 102 engages with the locking ring 1110 of the decoupling mechanism 170 and is forced to move a short distance in a direction opposite to that indicated by arrow A, resulting in a corresponding movement of the rod 508 of the actuator 102, to disengage the rod 508 from the plunger tip element 116 and hence decouple the manual drive mechanism (as described with reference to FIGS. 11A and 11B).

As the actuator 102 continues to rotate, each projection 510 on the actuator comes into axial alignment with a corresponding guide channel 1114 of the locking ring 1110 (as described with reference to FIGS. 11A and 11B). After this alignment occurs, further rotation of the actuator 102, in the rotational direction indicated by the arrows w, also induces corresponding rotation of the locking ring 1110 of the decoupling mechanism 170. The rotation of the locking ring 1110 of the decoupling mechanism 170 brings each guide channel 1114 into alignment with a corresponding projection 608 of the drive sleeve 120. Accordingly, once the rotation is complete, both the actuator 102 and the drive sleeve 120 have been released to move in the direction of arrow A. Accordingly, the rotation of the locking ring 1110 effectively engages and releases the assisted drive mechanism.

Moreover, as the actuator 102 rotates, the actuator 102 engages with the indicator ring 1010 of the indicator mechanism 160 to move the indicator ring back from the second indicator position to the first indicator position (as described with reference to FIG. 10). Thus, the visual indication 114 displayed through the aperture 139 provided in the barrel portion cover 130 changes from an arrow pointing in the rotational direction of arrow ω (as seen in FIG. 16) to an arrow pointing in the direction of arrow A (as seen in FIG. 18).

Referring to FIGS. 18 and 19, therefore, the action to rotate the actuator 102 decouples the manual drive mechanism, engages the assisted drive mechanism, and releases the actuator 102 and drive sleeve 120 for linear movement in the direction of arrow A (for subsequent delivery of the IOL).

It will be appreciated that, at this stage, a back-driving force is being induced on the rotatable element 124, by the compressed spring 126, that tends to urge rotation of the rotatable element 124 in a direction that would cause the rotatable element 124 to move linearly, relative to the threaded portion 118a (towards its distal end). It will be understood that such rotation would also cause rotation of the rotatable element 124 within the threaded part of the drive sleeve 120. Rotation of the rotatable element 124 within the threaded part of the drive sleeve 120 would, in turn, cause the drive sleeve 120 to move linearly towards the nozzle 112. However, while the drive sleeve 120 has been released for movement in the direction of arrow A, the components of the delivery device 100 are configured such that the force exerted by the spring 126 is not quite sufficient to overcome the cumulative forces opposing rotation of the rotatable element 124 (arising, for example, from the need to overcome the resistance to movement of the drive sleeve 120 provided by the resistance element 140, the friction inherent between the threaded surfaces, etc.). Thus, without a force being applied to the drive sleeve 120 in the direction of arrow A (e.g., by pressing the actuator 102), the spring force does not, by itself, induce movement of the various components of the delivery device 100 (although it will be appreciated that small movements may still occur as the inherent clearances between components will close as a result of a force being applied).

Other than the changes to the position of the actuator 102, locking ring 1110 of the decoupling mechanism 170, and indicator ring 1010 of the indicator mechanism 160, the various internal components of the delivery device 100 remain in essentially the same configuration as shown in FIGS. 16 and 17.

### Primed-and-Released State to Post-Delivery State

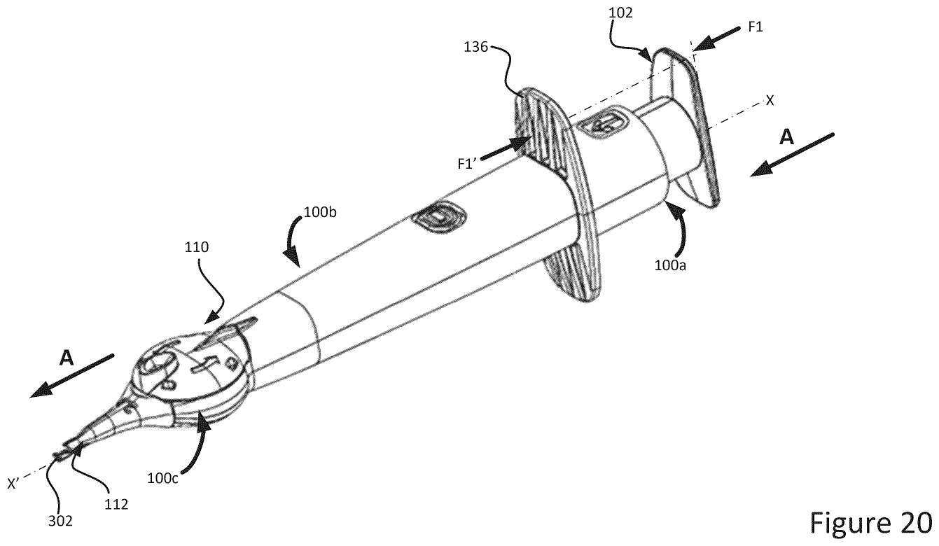

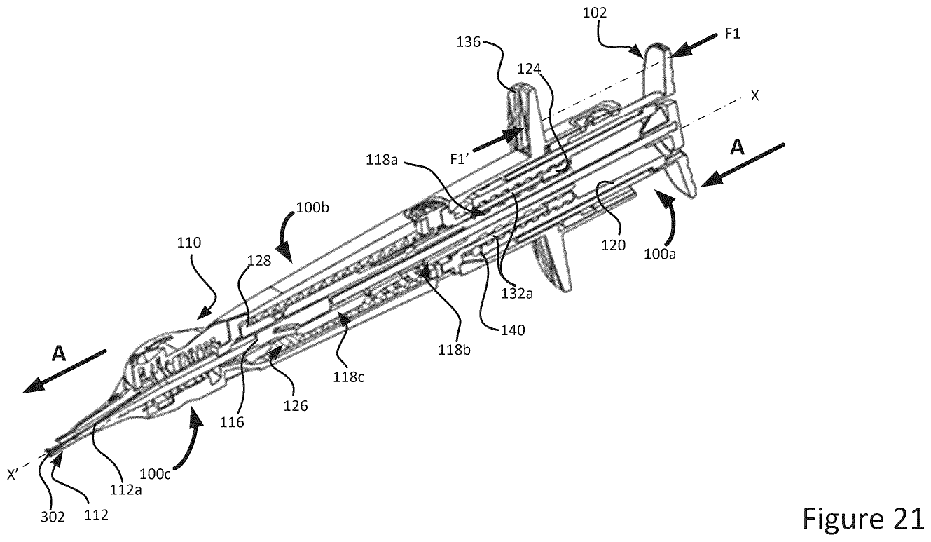

FIG. 20 is an illustration showing, a three-dimensional view of the delivery device 100 following an operation to deliver the IOL and hence change the state of the delivery device 100, from the primed-and-released state to a post-delivery state. FIG. 21 is an illustration showing a three-dimensional cross-sectional view of the delivery device in the configuration of FIG. 20, through a transverse plane perpendicular to a longitudinal axis of the delivery device.

Referring to FIGS. 20 and 21, to perform the operation to deliver the IOL (and hence change the state of the delivery device 100, from the primed-and-released state to a post-delivery state), the actuator 102 is pushed by a user in the direction of arrow A. Specifically, a user applies a force between the actuator 102 and the barrel portion 100b (as indicated by arrows F1-F1′), for example by the user pressing the actuator 102 with a thumb while retaining the finger flange 136 of the barrel portion 100b in position with two fingers.

As a result of the action to push the actuator 102, the actuator 102 also drives the plunger drive sleeve 120 in the direction of arrow A over the plunger guide portion 132a, against the resistance provided by the resistance element 140.

Thus, the rotatable element 124 begins to rotate within the drive sleeve 120, and around the threaded portion 118a of the plunger core 118, without axial movement of the rotatable element 124 because axial movement is inhibited by engagement of the rotatable element 124 against the distal end of the plunger guide portion 132a. The plunger core 118 thus begins to move axially, in the direction of arrow A, under the force exerted by the spring 126 against the spring retainer 128, without rotating because rotation of the plunger core 118 is inhibited by the keying features 410 of the central portion 118b. As this occurs the spring 126 decompresses as seen in FIG. 21.

As the plunger core 118 moves, the plunger core 118 also engages with the plunger tip element 116 to drive the plunger tip element 116 in the direction of arrow A. This engagement occurs as a result of the perimeter of the core engagement element reception apertures 406b located in the spring receiving portion 118b (described with reference to FIGS. 4A and 4B), engaging against the core engagement surface 304a of the core engagement element 304 (described with reference to FIG. 3).

Thus, the tip 302 of the plunger tip element 116 is initially driven into the OL/OVD capsule 110 of the delivery head 100c to engage with the IOL. As the plunger tip element 116 continues to move under the force exerted by the plunger core 118, the tip 302 of the plunger tip element 116 drives the IOL into the IOL/OVD conduit 112a of the nozzle 112, and then finally out of the nozzle 112 (e.g., for delivery into the eye of recipient).

Referring to FIGS. 20 and 21, therefore, the action to push the actuator 102 triggers the assisted drive mechanism to drive the plunger tip element 116, in the direction of arrow A, to deliver the IOL.

Possible material combinations that may, optionally, be used in fabrication of the delivery device will now be described by way of example only.

### Materials

It will be appreciated that the choice of material, especially the pairing of materials at the interface between different components that engage with, and move relative to one another, helps to ensure smooth and effective operation. Specifically, for smooth operation of the core mechanism, the material combinations are carefully selected to provide appropriate static and kinetic friction coefficients.

In more detail, the static friction coefficient is an indicator of when movement starts between two interfacing bodies, whereas the kinetic friction coefficient is an indicator of the force utilized to maintain movement at a desired speed of a design value.

Generally, there is a perception that the static friction coefficient will be larger than the kinetic friction coefficient. However, if this is the case, then movement can be subject to a sudden ‘jolt’ when a threshold value is reached. Following this jolt, movement starts and accelerates according to the difference between static and kinetic friction coefficients. The static friction coefficient may, nevertheless, be lower than the kinetic friction coefficient, in which case a slow (creeping) movement may start once the static threshold has been reached. In this case, however, full speed (as per the desired design value) may not be reached.

Contrastingly, if the static friction coefficient is equal (or near equal) to the kinetic friction coefficient, movement starts when the threshold is being reached, without a significant jolt and without significant creep. In this case, the speed at which the interface moves may be determined by the operator.

The friction coefficients available are largely dependent on the choice of material pairings, contact pressures, sliding speed, etc.

Accordingly, for smooth operation of the core mechanism, the material combinations are carefully selected to provide a relatively high kinetic friction coefficient (e.g., anywhere between 0.2 and 0.8) and a negligible drop in friction coefficient from static to kinetic. This is because of the operating environment (e.g., in an operating theatre) in which lubrication may not be permissible.

Extensive testing of materials has identified particularly suitable pairings of materials that may be used. Particularly advantageous material pairings include appropriate pairings of: a thermoplastic polyester elastomer (TPC-ET) (e.g., having a hardness rating of 55 on the Shore D hardness scale such as the a TPC-ET sold under the registered trademark Hytrel); an appropriate thermoplastic polymer (e.g., polypropylene or ‘PP’); a semi-crystalline thermoplastic with high mechanical strength and rigidity (e.g., Polyoxymethylene or ‘POM’); polycarbonate (PC); and Polyamide 12 (PA12).

Specifically, for the interface between the drive sleeve and rotatable element, it has been found that a kinetic friction coefficient between 0.4 and 0.8, and a static friction coefficient that is equal to or slightly (e.g., less than 10%) below the kinetic friction coefficient is particularly beneficial (e.g., to avoid a jolted start and the impression of a loss of control to an operator). This may be achieved, for example, by a delivery device having a drive sleeve formed of Hytrel® (or the like), and a rotatable element formed of POM (or the like), has been found to provide benefits in terms of operational performance at the interface between those components (e.g. for the rotatable element rotating smoothly in the drive sleeve).

Similarly, for the interface between the drive sleeve and the barrel portion cover, it has been found that a kinetic friction coefficient between 0.4 and 0.8, and a static friction coefficient that is equal to or slightly (e.g., less than 10%) below the kinetic friction coefficient is particularly beneficial (e.g., to avoid a jolted start and the impression of a loss of control to an operator). This may be achieved, for example, by a delivery device having a drive sleeve formed of Hytrel® (or the like), and a barrel portion cover formed of PP (or the like), has been found to provide benefits in terms of operational performance at the interface between those components (e.g. for the drive sleeve sliding smoothly into the cover).

Similarly, a delivery device having a drive sleeve formed of Hytrel® (or the like), and a plunger guide (‘post’) formed of PA12 (or the like), has been found to provide benefits in terms of operational performance at the interface between those components.

Similarly, for the interface between the plunger core and the rotatable element, it has been found that a relatively low kinetic coefficient of friction is beneficial (but not essential) to avoid unnecessary energy loss during delivery. In this case, a static friction coefficient that is marginally (e.g., less than 10%) greater than the kinetic friction coefficient is beneficial. This may be achieved, for example, by a delivery device having a plunger core formed of PC (or the like), and a rotatable element formed of POM (or the like), has been found to provide benefits in terms of operational performance at the interface between those components. It will be appreciated that this interface being biased towards stiction can beneficially help to reduce the risk of creep at other interfaces.

Similarly, for the interface between the plunger core and the plunger guide (‘post’), it has been found that a relatively low kinetic coefficient of friction is beneficial (but not essential) to avoid unnecessary energy loss during delivery. In this case, a static friction coefficient that is marginally (e.g., less than 10%) greater than the kinetic friction coefficient is beneficial. This may be achieved, for example, by a delivery device having a plunger core formed of PC (or the like), and a plunger guide (‘post’) formed of PA12 (or the like), has been found to provide benefits in terms of operational performance at the interface between those components. It will be appreciated that this interface being biased towards stiction can beneficially help to reduce the risk of creep at other interfaces.

Similarly, for the interface between the rotatable element and the plunger guide (‘post’), it has been found that a kinetic friction coefficient between 0.2 and 0.5, and a static friction coefficient that is slightly (e.g., less than 10%) below the kinetic friction coefficient is particularly beneficial (e.g., to avoid a jolted start and the impression of a loss of control to an operator). This may be achieved, for example, by a delivery device having a rotatable element formed of POM (or the like), and a plunger guide (‘post’) formed of PA12 (or the like), has been found to provide benefits in terms of operational performance at the interface between those components.

For the interface between the plunger guide ('post') and the resistance element (where this is an elastomer ring or the like), it has been found that a relatively high kinetic friction coefficient is beneficial. It will be appreciated the correct resistive force to push the friction element over the post arises from a combination of geometry and friction coefficients. In this case, a static friction coefficient that is equal to or slightly (e.g., less than 10%) below the kinetic friction coefficient is particularly beneficial (e.g., to avoid a jolted start and the impression of a loss of control to an operator). It will be appreciated that using a compression spring rather than an elastomer ring for the resistance element has the benefit that there will inherently be no jump from static to kinetic. Moreover, the resistive force will increase as the delivery progresses, mitigating some of the effects from interfaces where there is a noticeable step down in force from static to kinetic.

It will be appreciated that all or a subset of these material combinations may be used. Moreover there are other suitable material combinations that may be used that involve none, one, or more of the above materials for forming the abovementioned components.

### Modifications and Alternatives

A detailed example has been described above. As those skilled in the art will appreciate, a number of modifications and alternatives can be made to the above embodiments while still benefiting from the embodiments of the present disclosure.

For example, it will be appreciated that, while the above example is described in the context of an IOL delivery device, the technical principles described may be extended to procedures outside the IOL delivery space. For example, the principles could be applied in any situation in which a medical practitioner or other operator desires to deploy a potentially high force while simultaneously maintaining a high level of precision and control to administer a material or object. Even where the force or precision utilized is not particularly great, as those skilled in the art would recognize, the features described above have the potential to reduce the risk of repetitive strain injury in any similar procedure where an operator routinely administers a substance or object. In orthopedics for instance, similar procedures may include the injection of biomaterials, bone cement and hyaluronic acid. Similar applications may be found in spinal surgery and dentistry.

It can be seen that the exemplary plunger apparatus described above is configured to avoid any backlash in the chain of components from the actuator to the plunger tip and that there is essentially a one-to-one relationship between the actuator position and the plunger position. This means that the concept lends itself to any application in which the accurate positioning of something at the plunger tip by controlling the displacement of the actuator is desired. Other applications that may benefit from such control may be found in cardiology, e.g. for the placement of heart valves and stents, and neurology.

The presence of the preloaded spring, allowing the delivery device to be essentially free of backlash, means that there is the potential to use the same principles to provide a highly accurate instrument which could be used for the accurate and measurable delivery of precious drugs.

It will, nevertheless, be appreciated that the relation between actuator displacement and plunger core displacement need not be one to one. With appropriate design, the actuator and plunger core could be configured so that a small actuator displacement leads to a large plunger core displacement, which may be beneficial where the delivery device is used to deliver a certain quantity of substance fast. Conversely the actuator and plunger core could be configured such that a large actuator displacement leads to a small plunger core displacement (e.g. to provide more precise control at the plunger tip). The ratio between actuator and plunger core displacement can essentially be configured to be a fixed number, for example whereby a 10 mm actuator displacement results in a plunger core travel of, for example, 0.5 mm.

It will be appreciated that the device can be implemented either as a disposable device or as a reusable device.

It will be appreciated that while the use of the pre-compressed spring for providing the source of the power for driving the plunger is particularly beneficial, another source of mechanical power could be used either as an alternative to the spring or in addition to it. For example, a pneumatic actuator, or the like could be used. Such a pneumatic actuator may, for example, be powered by a source of compressed air, separate from the injector device, and ducted to the device by means of a flexible tube or the like. Compressed air is generally a readily available source of power, albeit that pneumatic actuators can sometimes be hard to control.

It will be appreciated that the spring may be arranged to be in a tensioned state for providing the source of the power for driving the plunger.

While, in the example above, the rotation of the back-driven nut is controlled by the movement of the axially controlled actuator, the rotation could be controlled in other ways. For example, rotation control could be provided by a rotary damper, such as an electro-mechanical damper. The operator in this scenario may, for example, be provided with a means to switch the damper on or off and/or a wheel attached to a variable resistor to control the speed of delivery.

It will be appreciated that, while the threads of the example described above are chosen such that the nut is back-drivable with respect to the threaded portion of the plunger core, a delivery device could be implemented in which the threads are configured such that the nut is not back-drivable with respect to the plunger. The rotation of the nut in an arrangement where it cannot be back-driven by the plunger can still be driven by an actuator as described for the above example albeit that the forces at play between nut and actuator are different as different faces of the thread profile will be engaged. For example, if the nut is not back-drivable with respect to the plunger core, then the force the operator exerts on the actuator will typically be the sum of the force utilized to move the actuator with respect of the housing and the force the actuator exerts on the thread of the nut in order to make the nut rotate.

A mechanism may be provided in the plunger apparatus for applying a force for actively rotating the nut and hence allow the plunger to be driven forward. While such a mechanism is particularly beneficial where the nut is not back-drivable it may also be used beneficially where the nut is back-drivable. For example, the rotation of the nut could be driven by electro-mechanical means, such as a geared motor or a stepper motor, where the surgeon is provided with a means to stop and start the rotation, and possibly to control the speed of delivery. An advantage of having a pre-compressed spring providing the main mechanical driving force in this case is that the motor only needs to provide a relatively low torque—i.e. enough to overcome the fiction.

Alternatively, or additionally, a torsion spring may be provided in the annular cavity which is preloaded when the device is assembled. This torsion spring may be configured to act on the nut to provide an active force for rotating the nut in a direction that will operate the plunger.

Various other modifications will be apparent to those skilled in the art and will not be described in further detail here.

1. A delivery device for deploying at least one object and/or material, the delivery device comprising:
a delivery portion comprising a nozzle, the delivery portion being arranged for delivery of the at least one object and/or material through the nozzle;
a barrel portion coupled to the delivery portion; and
a plunger portion at least partially received within the barrel portion, the plunger portion comprising:
a plunger tip element configured for movement in an axial direction relative to the barrel portion to engage with the at least one object and/or material, for delivery of the at least one object and/or material from the nozzle;
a plunger driver configured for movement in the axial direction relative to the barrel portion, and for engagement with the plunger tip element, to drive corresponding movement of the plunger tip element to deliver the at least one object and/or material;
a source of stored energy coupled to the plunger driver, the source of stored energy being arranged for providing a drive force to the plunger driver, when the stored energy is released, for driving the movement of the plunger driver and corresponding movement of the plunger tip element;
a release mechanism for providing a controlled release of the stored energy for providing the drive force for driving the movement of the plunger driver; and
an actuator configured for operation by an operator of the delivery device to control the release mechanism to induce the controlled release of the stored energy; wherein
wherein the plunger tip element and the plunger driver are mutually configured for operation between:
a disengaged arrangement in which the plunger driver is disengaged from the plunger tip element for the purpose of driving the corresponding movement of the plunger tip element; and
an engaged arrangement in which the plunger driver is engaged with the plunger tip element for the purpose of driving the corresponding movement of the plunger tip element.

2. A delivery device according to claim 1, wherein the actuator is configured for operation to change an arrangement of the plunger tip element and plunger driver from the disengaged arrangement to the engaged arrangement.

3. A delivery device according to claim 2, wherein the actuator is configured for operation to move the plunger tip element from a first position relative to the barrel portion to a second position relative to the barrel portion, whereby to change the arrangement of the plunger tip element and plunger driver from the disengaged arrangement to the engaged arrangement.

4. A delivery device according to claim 2, wherein the actuator is configured for operation to change the arrangement of the plunger tip element and plunger driver from the disengaged arrangement to the engaged arrangement by movement of the actuator in the axial direction, relative to the barrel portion, toward the nozzle.

5. A delivery device according to claim 1, wherein the release mechanism is configured for operation between:
a first configuration in which the release mechanism is inhibited from providing the controlled release of the stored energy to provide the drive force for driving the movement of the plunger driver; and
a second configuration in which the release mechanism is released from inhibition from providing the controlled release of the stored energy, whereby to allow for the controlled release of the stored energy to provide the drive force for driving the movement of the plunger driver.

6. A delivery device according to claim 1, further comprising a locking mechanism having a locking element configured for movement, when the plunger tip element and the plunger driver are in the disengaged arrangement, to change a configuration of the delivery device between:
a locking configuration in which the locking element engages with both the plunger tip element and the plunger driver to inhibit movement of the plunger tip element and the plunger driver in the axial direction; and
an unlocked configuration in which the locking element is disengaged from both the plunger tip element and the plunger driver, to release the plunger tip element and the plunger driver from inhibition by the locking element, whereby to allow for movement of the plunger tip element and the plunger driver in the axial direction.

7. A delivery device according to claim 1, wherein the actuator is configured for operation by an operator through a sequence of operations to control operation of the delivery device to perform delivery of the at least one object and/or material through the nozzle, the sequence of operations including a plurality of different types of actuator movement, wherein the delivery device further comprises an indicator mechanism configured to:
display a first indication of a first type of actuator movement of the plurality of different types of actuator movement, utilized in a first operation of the sequence of operations, and
to display a second indication of a second different type of actuator movement of the plurality of different types of actuator movement, utilized in a second operation of the sequence of operations, following the first operation, as a result of movement of the actuator to perform the first operation of the sequence of operations.

8. A delivery device for deploying at least one object and/or material, the delivery device comprising:
a delivery portion comprising a nozzle, the delivery portion being arranged for delivery of the at least one object and/or material through the nozzle;
a barrel portion coupled to the delivery portion; and
a plunger portion at least partially received within the barrel portion, the plunger portion comprising:
a plunger tip element configured for movement in an axial direction relative to the barrel portion to engage with the at least one object and/or material, for delivery of the at least one object and/or material from the nozzle;
a plunger driver configured for movement in the axial direction relative to the barrel portion, and for engagement with the plunger tip element, to drive corresponding movement of the plunger tip element to deliver the at least one object and/or material;
a source of stored energy coupled to the plunger driver, the source of stored energy being arranged for providing a drive force to the plunger driver, when the stored energy is released, for driving the movement of the plunger driver and corresponding movement of the plunger tip element;
a release mechanism for providing a controlled release of the stored energy for providing the drive force for driving the movement of the plunger driver; and
an actuator configured for operation by an operator of the delivery device to control the release mechanism to induce the controlled release of the stored energy;
wherein the release mechanism and the plunger driver are mutually configured for operation between:
a first configuration in which the release mechanism is inhibited from providing the controlled release of the stored energy to provide the drive force for driving the movement of the plunger driver; and
a second configuration in which the release mechanism is released from inhibition from providing the controlled release of the stored energy, whereby to allow for the controlled release of the stored energy to provide the drive force for driving the movement of the plunger driver.

9. A delivery device according to claim 8, wherein the actuator is configured for operation to change a configuration of the release mechanism and plunger driver from the first configuration to the second configuration.

10. A delivery device according to claim 8, wherein the actuator is configured for operation to change the configuration of the release mechanism and plunger driver from the first configuration to the second configuration by rotation of the actuator about a longitudinal axis of the barrel portion.

11. A delivery device according to claim 8, wherein the actuator is configured for operation by an operator of the delivery device to control the release mechanism to induce the controlled release of the stored energy, when the release mechanism and the plunger driver are in the second configuration, by movement of the actuator in the axial direction, relative to the barrel portion, toward the nozzle.

12. A delivery device according to claim 8, wherein the release mechanism comprises a drive portion and a rotatable element that are mutually configured for rotation of the rotatable element relative to the drive portion, about an axis of rotation that is parallel to the axial direction, whereby rotation of the rotatable element through a given cumulative rotational displacement provides the controlled release of the stored energy for driving the movement of the plunger driver, in the axial direction relative to the barrel portion, under the force provided by the source of stored energy, for a longitudinal displacement that is dependent on the cumulative rotational displacement.

13. A delivery device according to claim 12, wherein the actuator is configured for axial movement, by the operator, relative to the barrel portion to induce corresponding axial movement of the drive portion, wherein the corresponding axial movement of the drive portion induces the rotation of the rotatable element relative to the drive portion to provide the controlled release of the stored energy.

14. A delivery device according to claim 13, further comprising at least one element for providing a preconfigured resistance between the drive portion and the barrel portion for resisting axial movement of the drive portion relative to the barrel portion whereby the drive portion is moveable by an operator applying a substantially uniform force that is sufficient for overcoming the preconfigured resistance.

15. A delivery device according to claim 14, wherein the resistance element comprises a spring.

16. A delivery device according to claim 13, wherein the drive portion and the rotatable element each have a respective threaded surface, wherein the threaded surfaces of the drive portion and the rotatable element are configured for mutual engagement whereby the rotatable element is configured for the rotation relative to the drive portion, as the drive portion is moved in the axial direction by the actuator, whereby to provide the controlled release of the stored energy.

17. A delivery device according to claim 16, wherein the threaded surfaces of the drive portion and the rotatable element are mutually configured such that the rotatable element is back-drivable with respect to the drive portion.

18. A delivery device according to claim 12, wherein the drive portion comprises a tubular sleeve, and the tubular sleeve and rotatable element are mutually configured for rotation of the rotatable element within the tubular sleeve.

19. A delivery device according to claim 18, wherein the plunger driver and the rotatable element each have a respective threaded surface, wherein the threaded surfaces of the plunger driver and the rotatable element are configured for mutual engagement whereby the rotatable element is configured for rotation relative to the plunger driver as the rotatable element rotates relative to the sleeve to provide the controlled release of the stored energy.

20. A delivery device according to claim 19, wherein the threaded surfaces of the plunger driver and the rotatable element are mutually configured such that the rotatable element is back-drivable with respect to the plunger driver.

21. A delivery device according to claim 12, wherein the drive portion comprises a cylindrical threaded portion, and the cylindrical threaded portion and rotatable element are mutually configured for rotation of the rotatable element about the cylindrical portion.

22. A delivery device according to claim 8, wherein the source of stored energy is a source of stored mechanical energy.

23. A delivery device according to claim 22, wherein the source of stored mechanical energy comprises a spring that is arranged for storing elastic potential energy prior to operation of the delivery device for providing the drive force for driving the movement of the plunger driver.

24. A delivery device according to claim 23, wherein the spring is arranged to be in a compressed state for storing the elastic potential energy prior to operation of the delivery device for providing the drive force for driving movement of the plunger driver.

25. A delivery device according to claim 12, wherein the drive portion and the rotatable element are formed of materials that provide a kinetic friction coefficient of between 0.4 and 0.8 inclusive at an interface between the drive portion and the rotatable element.

26. A delivery device according to claim 12, wherein the plunger driver and the rotatable element are formed of materials that provide a kinetic friction coefficient of between 0.2 and 0.5 inclusive at an interface between the plunger driver and the rotatable element.

27. A delivery device according to claim 12, wherein the drive portion is configured for slidable movement within the barrel portion, and the drive portion and barrel portion are formed of materials that provide a kinetic friction coefficient of between 0.4 and 0.8 inclusive at an interface between the drive portion and barrel portion.
